(19) United States
(12) Patent Application Publication
(10) Pub. No.: US 2010/0076328 A1
(43) Pub. Date: Mar. 25, 2010

(54) PULSE WAVE MEASUREMENT ELECTRODE UNIT AND PULSE WAVE MEASUREMENT DEVICE

(75) Inventors: Naomi Matsumura, Takatsuki-shi (JP); Yukiya Sawanoi, Nara (JP); Toshiyuki Iwahori, Osaka (JP)

Correspondence Address: MORRISON & FOERSTER LLP, 1650 TYSONS BOULEVARD, SUITE 400, MCLEAN, VA, 22102, US

(73) Assignee: OMRON HEALTHCARE CO., LTD., KYOTO (JP)

(21) Appl. No.: 12/516,307

(22) PCT Filed: Nov. 12, 2007
(86) PCT No.: PCT/JP2007/071910

§ 371 Date: Jul. 30, 2009

(30) Foreign Application Priority Data
Dec. 1, 2006 (JP) 2006-325914

(51) Int. Cl. A61B 5/024

(52) U.S. Cl. 600/500

(57) ABSTRACT

A pulse wave measurement electrode unit includes an electrode group and a supporting member. The electrode group includes a first electrode portion including a first current application electrode and a first voltage measurement electrode, and a second electrode portion, positioned spaced apart from the first electrode portion, including a second current application electrode and a second voltage measurement electrode. The supporting member supports the electrode group such that contacting surfaces of the electrodes with respect to a living body are arranged substantially on an identical plane. The electrodes are arranged lined in a direction an artery extends when the pulse wave measurement electrode unit is applied to the living body. According to such a configuration, a pulse wave measurement electrode unit having a simple configuration and enabling high precision volume pulse wave measurement can be provided.

### REFERENCE TO RELATED APPLICATIONS

This application is a national stage application under 35 USC 371 of International Application No. PCT/JP2007/071910, filed Nov. 12, 2007, which claims the priority of Japanese Patent Application No. 2006-325914, filed Dec. 1, 2006, the contents of which prior applications are incorporated herein by reference.

### FIELD OF THE INVENTION

The present invention relates to a pulse wave measurement electrode unit to be attached to a living body to acquire a volume pulse wave of an artery by measuring a fluctuation of a biological impedance, and a pulse wave measurement device equipped with the same.

### BACKGROUND OF THE INVENTION

It is very important to measure a pulse wave of an artery of a subject in knowing a health condition of the subject. In recent years, measurement of the pulse wave of the artery of the subject is frequently being performed to understand a change and the like of a cardiac load and a hardness of the artery. A blood pressure value (systolic blood pressure value and diastolic blood pressure value), which usability is widely recognized as a typical index of health management from the related art, is also derived from the pulse wave of the artery. The pulse wave measurement device is a device for measuring the pulse wave of the artery serving as important biological information, and further utilization in fields of early detection and prevention, treatment and the like of circulatory system diseases is being anticipated.

A volume pulse wave shows a periodic volume fluctuation of a blood vessel involved in a beating of a heart as a wave motion, but in this regards, if the volume fluctuation of the blood vessel is observed at least with a time difference, it can be referred to as the volume pulse wave regardless of such a temporal resolution in the present specification. It should be recognized that the temporal resolution needs to be high in order to accurately capture the volume pulse wave included in one beat.

The term “pulse wave measurement device” used herein refers to the overall device that has at least a function of measuring the volume pulse wave, and is not limited to those which output the measured volume pulse wave as is for a measurement result, but also includes those which calculate or measure other specific indices based on the measured volume pulse wave, and output only the index obtained as a result for the measurement result. Therefore, the volume pulse wave measurement device includes, for example, a blood pressure measurement device that acquires the volume pulse wave in a measurement process but outputs only the blood pressure value without outputting the volume pulse wave itself, or the like.

The pulse wave measurement device capable of noninvasively measuring the pulse wave of the artery without giving pain to the subject is classified into the following five categories based on a difference in a measurement method.

A pulse wave measurement device based on a first measurement method includes a cuff for compressing the artery by being wrapped around a measuring site of the living body, and measures a pressure pulse wave of the artery by detecting with a pressure sensor and the like the fluctuation in the cuff pressure of when the measuring site is compressed using the cuff. However, in the pulse wave measurement device based on the first measurement method, a large difference in a compression force with respect to the measuring site is created between an end and a central part of the cuff when compressing the measuring site with the cuff, and thus it is difficult to evenly compress the measuring site, and high precision pulse wave measurement is difficult. When using the site where plural arteries are running such as a wrist for the measuring site, the pulse waves of the plural arteries are averaged and detected, and thus high precision pulse wave measurement is difficult.

A pulse wave measurement device based on a second measurement method includes a pressure sensor having a planar pressure sensitive surface, and a pushing mechanism for pushing the pressure sensor against the measuring site of the living body, which device pushes the pressure sensor against the measuring site using the pushing mechanism until a flat portion forms at a blood vessel wall of the artery, and measures the pressure pulse wave of the artery based on the pressure information detected by the pressure sensor at the time. Such a measurement method is generally referred to as a tonometry method. However, in the pulse wave measurement device using the tonometry method, a flat portion always needs to be formed at the blood vessel wall of the artery when measuring the pulse wave, and measurement accuracy significantly degrades if this condition is not satisfied. Thus, only the site of the living body where the artery is running at a position where a depth from a body surface is relatively shallow can be used for the measuring site. The pressure sensor also needs to be accurately positioned and pushed against the skin located immediately above the artery, which complicates and enlarges a configuration of the device.

A pulse wave measurement device based on a third measurement method includes an ultrasonic wave sensor, and measures the volume pulse wave of the artery using the ultrasonic sensor. However, also in the pulse wave measurement device using the third measurement method, only the site of the living body where the artery is running at the position where the depth from the body surface is relatively shallow can be used for the measuring site. Furthermore, there is a problem that the device is very expensive and large in size.

A pulse wave measurement device based on a fourth measurement method includes a light emitting element and a light receiving element, and measures the volume pulse wave of the artery by detecting the fluctuation in blood tissue volume through an optical method. However, in the pulse wave measurement device using the fourth measurement method, light emitted from the light emitting element needs to be accurately captured by the light receiving element, which arises problems such as a need to enhance positioning accuracy between the light emitting element and the light receiving element.

A pulse wave measurement device based on a fifth measurement method includes a measurement electrode including a plurality of electrodes, which device brings the measurement electrode into contact with the measuring site of the living body, detects the fluctuation in blood tissue volume as fluctuation in biological impedance, and measures the volume pulse wave of the artery therefrom. The pulse wave measurement device using the fifth measurement method can be inexpensively manufactured with a relatively easy configuration, and has a merit in that a biological information measurement electrode widely used in fields of electrocardiographic measurement, body fat measurement and the like can be applied for the measurement electrode with the configuration substantially unchanged. Furthermore, the device also has an advantage in that any site can be used for the measuring site as long as it is the site of the living body where the artery is running, and thus a degree of freedom in the pulse wave measurement becomes very high. Based on the reasons above, the pulse wave measurement device using the biological impedance method or the fifth measurement method is particularly receiving attention.

A document disclosing the pulse wave measurement device using the biological impedance method or the fifth measurement method and the pulse wave measurement electrode unit used therefor includes, for example, Japanese Unexamined Patent Publication No. 2004-242851 (Patent Document 1). In the pulse wave measurement electrode unit disclosed in Patent Document 1, a pair of electrode portions for pulse wave measurement are arranged on an outer surface of a supporting member, which pair of electrode portions are arranged in a direction orthogonal to an extending direction of the artery (i.e., running direction of the artery) when the supporting member is attached to the measuring site of the living body. In other words, the artery is sandwiched in the direction orthogonal to the running direction of the artery by the pair of electrode portions, and the electrode portion is not attached to a skin immediately above the artery.

[Patent Document 1] Japanese Unexamined Patent Publication No. 2004-242851

### SUMMARY OF THE INVENTION

However, when pulse wave measurement is performed using a pulse wave measurement electrode unit and a pulse wave measurement device equipped with the same having a configuration disclosed in Patent Document 1, a great amount of a body tissue portion other than an artery is included in a measuring site or a site where a constant current applied for pulse wave measurement passes, and an impedance fluctuation at the body tissue portion other than the artery superimposes the measured volume pulse wave as an error component of volume pulse wave measurement. Therefore, high precision pulse wave measurement becomes difficult. If an electrode length in a direction parallel to a running direction of the artery is made long so that a long artery portion where the constant current passes can be ensured, the body tissue portion other than the artery at the measuring site where the constant current passes also increases, and thus measurement accuracy still does not improve.

Therefore, in view of solving the above-described problems, it is an object of the present invention to enable volume pulse wave measurement of high precision in a pulse wave measurement electrode unit to be attached to a living body to acquire the volume pulse wave of the artery by measuring the fluctuation of the biological impedance, and the pulse wave measurement device quipped with the same.

According to one aspect of the present invention, a pulse wave measurement electrode unit is attached to a living body for acquiring a volume pulse wave of an artery by measuring a fluctuation of a biological impedance; and the pulse wave measurement electrode unit includes: an electrode group including a pair of current application electrodes and a pair of voltage measurement electrodes, and brought into contact with a body surface of the living body during measurement; and a supporting member for supporting the electrode group. The electrode group includes a first electrode portion including one of the pair of current application electrodes and one of the pair of voltage measurement electrodes, and a second electrode portion, positioned spaced apart from the first electrode portion, including the other one of the pair of current application electrodes and the other one of the pair of voltage measurement electrodes. The supporting member supports the electrode group such that a contacting surface with respect to the living body of the first electrode portion and a contacting surface with respect to the living body of the second electrode portion are arranged substantially on an identical plane, and the first electrode portion and the second electrode portion are arranged lined in a direction the artery extends when the pulse wave measurement electrode unit is attached to the living body.

In the pulse wave measurement electrode unit according to one aspect of the present invention, the first electrode portion may include a single electrode used as one of the pair of current application electrodes and one of the pair of voltage measurement electrodes; and the second electrode portion may include a single electrode used as the other one of the pair of current application electrodes and the other one of the pair of voltage measurement electrodes, or the first electrode portion may include two electrodes in which one of the pair of current application electrodes and one of the pair of voltage measurement electrodes are separated and independent; and the second electrode portion may include two electrodes in which the other one of the pair of current application electrodes and the other one of the pair of voltage measurement electrodes are separated and independent.

In the pulse wave measurement electrode unit according to one aspect of the present invention, the contacting surface of each of the pair of current application electrodes and the pair of voltage measurement electrodes with the body surface of the living body preferably has a substantially rectangular shape in plan view; and in this case, a length of the current application electrode in a direction intersecting a direction the first electrode portion and the second electrode portion are lined is preferably equal to or less than a length of the voltage measurement electrode in the direction intersecting the direction the first electrode portion and the second electrode portion are lined.

The pulse wave measurement electrode unit according to one aspect of the present invention includes: the pulse wave measurement electrode unit according to one aspect of the present invention; a constant current supply section for supplying a constant current between the pair of current application electrodes; an impedance measurement section for measuring a fluctuation of a biological impedance by detecting a potential difference created between the pair of voltage measurement electrodes; and a volume pulse wave acquiring unit for acquiring a volume pulse wave of an artery based on information obtained by the impedance measurement section.

In accordance with the pulse wave measurement electrode unit according to another aspect of the present invention, in the pulse wave measurement electrode unit according to one aspect of the present invention, the electrode group is arranged in plural sets; and the supporting member supports the plural sets of electrode groups such that the plural sets of electrode groups are arranged lined in a direction intersecting a direction the first electrode portion and the second electrode portion are lined.

The pulse wave measurement electrode unit according to another aspect of the present invention includes: the pulse wave measurement electrode unit according to another aspect of the present invention; a first electrode portion selecting unit for switchably selecting a specific first electrode portion of a plurality of first electrode portions included in the pulse wave measurement electrode unit; a second electrode portion selecting unit for switchably selecting a specific second electrode portion of a plurality of second electrode portions included in the pulse wave measurement electrode unit; a constant current supply section for supplying constant current between current application electrodes included in the specific first electrode portion selected by the first electrode portion selecting unit and the specific second electrode portion selected by the second electrode portion selecting unit; an impedance measurement section for measuring a fluctuation of a biological impedance by detecting a potential difference created between voltage measurement electrodes included in the specific first electrode portion selected by the first electrode portion selecting unit and the specific second electrode portion selected by the second electrode portion selecting unit; and a volume pulse wave acquiring unit for acquiring a volume pulse wave of an artery based on information obtained in the impedance measurement section.

The pulse wave measurement electrode unit according to another aspect of the present invention includes: the pulse wave measurement electrode unit according to another aspect of the present invention; a first electrode portion current application electrode selecting unit for switchably selecting a current application electrode included in a specific first electrode portion of a plurality of first electrode portions included in the pulse wave measurement electrode unit; a first electrode portion voltage measurement electrode selecting unit for switchably selecting a voltage measurement electrode included in a specific first electrode portion of a plurality of first electrode portions included in the pulse wave measurement electrode unit; a second electrode portion current application electrode selecting unit for switchably selecting a current application electrode included in a specific second electrode portion of a plurality of second electrode portions included in the pulse wave measurement electrode unit; a second electrode portion voltage measurement electrode selecting unit for switchably selecting a voltage measurement electrode included in a specific second electrode portion of a plurality of second electrode portions included in the pulse wave measurement electrode unit; a constant current supply section for supplying a constant current between current application electrodes included in the specific first electrode portion selected by the first electrode portion current application electrode selecting unit and current application electrodes included in the specific second electrode portion selected by the second electrode portion current application electrode selecting unit; an impedance measurement section for measuring a fluctuation of a biological impedance by detecting a potential difference created between voltage measurement electrodes included in the specific first electrode portion selected by the first electrode portion voltage measurement electrode selecting unit and between voltage measurement electrodes included in the specific second electrode portion selected by the second electrode portion voltage measurement electrode selecting unit; and a volume pulse wave acquiring unit for acquiring a volume pulse wave of an artery based on information obtained in the impedance measurement section.

The pulse wave measurement device according to all aspects of the present invention preferably further includes a compression mechanism for pressing a body surface of a living body to compress the artery, in which case, the pulse wave measurement electrode unit according to all aspects of the present invention is preferably arranged on a compression acting surface of the compression mechanism. In this case, the compression mechanism preferably includes a first compression mechanism for pressing a portion arranged with the first electrode portion and the second electrode portion of the supporting member towards the living body, and a second compression mechanism for pressing a portion positioned between the first electrode portion and the second electrode portion of the supporting member towards the living body.

The pulse wave measurement device according to all aspects of the present invention may further include an ejection wave/reflected wave acquiring unit for acquiring at least one of an ejection wave or a reflected wave based on information of the volume pulse wave obtained in the volume pulse wave acquiring unit.

The pulse wave measurement device according to all aspects of the present invention may further include a compression mechanism for pressing a body surface of a living body to compress the artery; a compression force detection unit capable of detecting a compression force on the artery by the compression mechanism; and a blood pressure value acquiring unit for acquiring a diastolic blood pressure value and a systolic blood pressure value based on information of the volume pulse wave obtained in the volume pulse wave acquiring unit and information of the compression force obtained in the compression force detection unit.

The pulse wave measurement device according to all aspects of the present invention may further include a compression mechanism for pressing a body surface of a living body to compress the artery; a compression force control unit for servo controlling a compression force on the artery by the compression mechanism based on information of the volume pulse wave obtained in the volume pulse wave acquiring unit; a compression force detection unit capable of detecting a compression force on the artery by the compression mechanism; and a blood pressure value acquiring unit for acquiring a diastolic blood pressure value and a systolic blood pressure value based on information of the compression force obtained in the compression force detection unit.

A volume pulse wave can be measured at high precision by using a pulse wave measurement electrode unit and a pulse wave measurement device equipped with the same according to the present invention.

### BRIEF DESCRIPTION OF THE DRAWINGS

FIG. 1 is a function block diagram showing a configuration of a pulse wave measurement device according to a first embodiment of the present invention.

FIG. 2 is a schematic perspective view of a pulse wave measurement electrode unit according to the first embodiment of the present invention.

FIG. 3 is a plan view showing a state in which the pulse wave measurement electrode unit is attached to a wrist in the pulse wave measurement device according to the first embodiment of the present invention.

FIG. 4 is a schematic cross-sectional view taken along line IV-IV shown in FIG. 3.

FIG. 5 is a flowchart showing a processing procedure of the pulse wave measurement device according to the first embodiment of the present invention.

FIG. 6 is a graph showing a waveform of a volume pulse wave actually obtained by a pulse wave measurement device 100A according to the first embodiment of the present invention.

FIG. 7A is an electrode layout diagram showing one example where an electrode layout of the pulse wave measurement electrode unit is variously changed in the pulse wave measurement device according to the first embodiment of the present invention.

FIG. 7B is a graph showing the waveform of the volume pulse wave obtained when pulse wave measurement is performed using the electrode layout shown in FIG. 7A.

FIG. 8A is an electrode layout diagram showing another example where the electrode layout of the pulse wave measurement electrode unit is variously changed in the pulse wave measurement device according to the first embodiment of the present invention.

FIG. 8B is a graph showing the waveform of the volume pulse wave obtained when the pulse wave measurement is performed using the electrode layout shown in FIG. 8A.

FIG. 9A is an electrode layout diagram showing another further example where the electrode layout of the pulse wave measurement electrode unit is variously changed in the pulse wave measurement device according to the first embodiment of the present invention.

FIG. 9B is a graph showing the waveform of the volume pulse wave obtained when the pulse wave measurement is performed using the electrode layout shown in FIG. 9A.

FIG. 10A is an electrode layout diagram showing another further example where the electrode layout of the pulse wave measurement electrode unit is variously changed in the pulse wave measurement device according to the first embodiment of the present invention.

FIG. 10B is a graph showing the waveform of the volume pulse wave obtained when the pulse wave measurement is performed using the electrode layout shown in FIG. 10A.

FIG. 11 is a function block diagram showing a configuration of a pulse wave measurement device according to a second embodiment of the present invention.

FIG. 12 is a schematic perspective view of a cuff of the pulse wave measurement device according to the second embodiment of the present invention.

FIG. 13 is a cross-sectional view showing a state in which the cuff of the pulse wave measurement device according to the second embodiment of the present invention is attached to the wrist.

FIG. 14 is a function block diagram showing another configuration example of the pulse wave measurement device according to the second embodiment of the present invention.

FIG. 15 is a view showing a modification of the configuration example shown in FIG. 14.

FIG. 16 is a function block diagram showing a configuration of a pulse wave measurement device according to a third embodiment of the present invention.

FIG. 17 is a function block diagram showing a configuration of a pulse wave measurement device according to a fourth embodiment of the present invention.

FIG. 18 is a view showing one example of a positional relationship between an electrode and a radial artery when the pulse wave measurement electrode unit is attached to the wrist in the pulse wave measurement device according to the fourth embodiment of the present invention.

FIG. 19 is a view showing another example of the positional relationship between the electrode and the radial artery when the pulse wave measurement electrode unit is attached to the wrist in the pulse wave measurement device according to the fourth embodiment of the present invention.

FIG. 20 is a view showing another further example of the positional relationship between the electrode and the radial artery when the pulse wave measurement electrode unit is attached to the wrist in the pulse wave measurement device according to the fourth embodiment of the present invention.

FIG. 21 is a flowchart showing a processing procedure of the pulse wave measurement device according to the fourth embodiment of the present invention.

FIG. 22 is a view showing another further example of the positional relationship between the electrode and the radial artery when the pulse wave measurement electrode unit is attached to the wrist in the pulse wave measurement device according to the fourth embodiment of the present invention.

FIG. 23 is a function block diagram showing a configuration of a pulse wave measurement device according to a fifth embodiment of the present invention.

FIG. 24 is a flowchart showing a processing procedure of the pulse wave measurement device according to the fifth embodiment of the present invention.

FIG. 25 is a function block diagram showing a configuration of a pulse wave measurement device according to a sixth embodiment of the present invention.

FIG. 26 is a flowchart showing a processing procedure of the pulse wave measurement device according to the sixth embodiment of the present invention.

FIG. 27 is a function block diagram showing a configuration of a pulse wave measurement device according to a seventh embodiment of the present invention.

FIG. 28 is a flowchart showing a processing procedure of the pulse wave measurement device according to the seventh embodiment of the present invention.

### DETAILED DESCRIPTION OF THE INVENTION

Hereinafter, embodiments of the present invention will be described in detail with reference to the drawings. In the embodiments described below, a case will be illustrated and described where a wrist is a measuring site, and where the present invention is applied to a pulse wave measurement device capable of noninvasively measuring a volume pulse wave of a radial artery extending in the wrist.

### First Embodiment

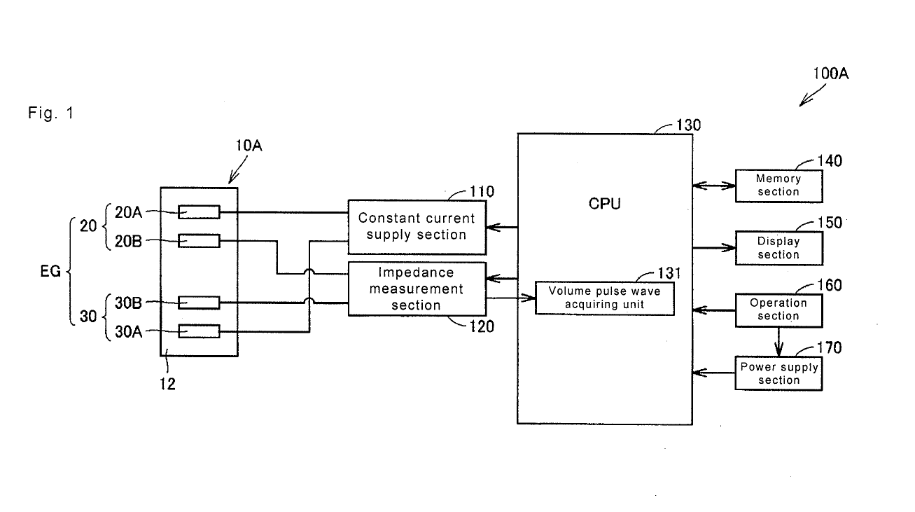

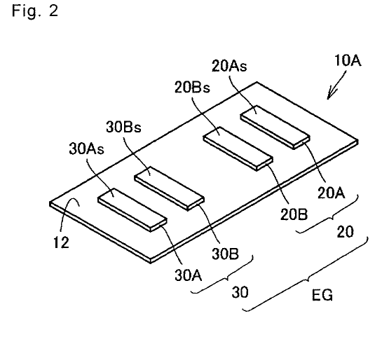

FIG. 1 is a function block diagram showing a configuration of a pulse wave measurement device according to a first embodiment of the present invention, and FIG. 2 is a schematic perspective view of a pulse wave measurement electrode unit according to the present embodiment. A configuration of a pulse wave measurement device 100A according to the present embodiment and an outer appearance structure of a pulse wave measurement electrode unit 10A will be described with reference to FIGS. 1 and 2.

As shown in FIG. 1, the pulse wave measurement device 100A according to the present embodiment mainly includes a pulse wave measurement electrode unit 10A, a constant current supply section 110, an impedance measurement section 120, a CPU 130, a memory section 140, a display section 150, an operation section 160, and a power supply section 170.

As shown in FIGS. 1 and 2, the pulse wave measurement electrode unit 10A is attached to a living body to measure a fluctuation of a biological impedance, and includes a supporting member 12 and an electrode group EG including a plurality of electrodes 20A, 20B, 30A, and 30B. In particular, the pulse wave measurement electrode unit 10A according to the present embodiment has a shape suited for attachment to a wrist of a subject, and detects a blood tissue volume fluctuation of the radial artery as the biological impedance fluctuation to acquire the volume pulse wave of the radial artery extending through the wrist to which it is attached.

As shown in FIG. 2, the supporting member 12 is configured, for example, by a sheet-shaped member, and has the electrode group EG on a main surface, which is positioned on the wrist side when attached to the wrist. The electrodes 20A, 20B, 30A, and 30B configuring the electrode group EG are exposed at the main surface of the supporting member 12, and can be brought into contact with the surface of the wrist when the pulse wave measurement electrode unit 10A is attached to the wrist.

As shown in FIGS. 1 and 2, the electrode group EG includes a first electrode portion 20 and a second electrode portion 30 arranged with a predetermined distance from the first electrode portion 20. The first electrode portion 20 includes two electrodes that are separated and independent, and includes a first current application electrode 20A, which is one of a pair of current application electrodes, and a first voltage measurement electrode 20B, which is one of a pair of voltage measurement electrodes. The second electrode portion 30 includes two electrodes that are separated and independent, and includes a second current application electrode 30A, which is the other of the pair of current application electrodes, and a second voltage measurement electrode 30B, which is the other of the pair of voltage measurement electrodes.

Such electrodes 20A, 20B, 30A, and 30B are respectively formed in a substantially rectangular shape in plan view as shown in the figure. The pair of voltage measurement electrodes 20B, 30B are sandwiched by the pair of current application electrodes 20A, 30A, so that the electrodes 20A, 20B, 30A, and 30B are arranged aligned in a line on the supporting member 12. The supporting member 12 supports each of the electrodes 20A, 20B, 30A, and 30B so that the aligning direction of each of the electrodes 20A, 20B, 30A, and 30B coincides with an extending direction of the radial artery extending through the wrist when the pulse wave measurement electrode unit 10A is attached to the wrist.

As shown in FIG. 2, in the pulse wave measurement electrode unit 10A according to the present embodiment, contacting surfaces 20As, 20Bs, 30As, and 30Bs with the wrist of the electrodes 20A, 20B, 30A, and 30B are positioned on an identical plane. The term “identical plane” herein includes both identical flat surface and identical curved surface. When configured such that the contacting surfaces 20As, 20Bs, 30As, and 30Bs are positioned on the same curved surface, the curved surface is, in particular, preferably a curved surface that curves only in a direction substantially orthogonal to the aligning direction of the electrodes 20A, 20B, 30A, and 30B, but may also be a curved surface that curves only in a direction parallel to the aligning direction of the electrodes 20A, 20B, 30A, and 30B.

The supporting member 12 is made from an insulative resin member and the like. The supporting member 12 preferably has a rigidity to an extent the supporting member 12 does not deflect by a tension of a skin in the attached state, and the contacting surfaces 20As, 20Bs, 30As, and 30Bs with the wrist of the electrodes 20A, 20B, 30A, and 30B are prevented from not being positioned on an identical plane. Therefore, the supporting member 12 is preferably made from a hard resin member, a resin member having an appropriate flexibility of a range it cannot be bent by the tension of the skin, or the like. However, if some kind of auxiliary member (e.g., cuff as shown in second embodiment to be hereinafter described) for holding the supporting member 12 exists, a resin member of film-form having poor rigidity and being bent by the tension of the skin by itself, and the like may be used.

The pair of current application electrodes 20A, 20B and the pair of voltage measurement electrodes 30A, 30B are configured by conductive members. Such electrodes 20A, 20B, 30A, and 30B are electrodes that are all brought into contact with the wrist, and thus are preferably made from a material excelling in biocompatibility. From such a point of view, the electrodes 20A, 20B, 30A, and 30B preferably use a metal member such as Ag (silver)/AgCl (silver chloride) and the like, which is the electrode member used in electrocardiogram measurement and body fat measurement.

As shown in FIG. 1, the pair of current application electrodes 20A, 30A are respectively electrically connected to the constant current supply section 110. The constant current supply section 110 is means for supplying constant current to the pair of current application electrodes 20A, 30A, and generates a constant current having a frequency of about 50 kHz and a current amount of about 500 μA between the pair of current application electrodes 20A, 30A.

The pair of voltage measurement electrodes 20B, 30B are respectively electrically connected to the impedance measurement section 120. The impedance measurement section 120 is means for measuring the fluctuation of the biological impedance between the electrodes 20B, 30B by detecting a potential difference created between the pair of voltage measurement electrodes 20B, 30B. The impedance measurement section 120 includes processing circuits such as an analog filter circuit, a rectifier circuit, an amplification circuit, and an ND (analog/digital) conversion circuit, and digitizes the biological impedance detected as an analog value and outputs the same.

As shown in FIG. 1, the CPU 130 is means for controlling the entire pulse wave measurement device 100A. The memory section 140 is configured by a ROM and a RAM, and is means for storing a program for causing the CPU 130 and the like to execute the processing procedure for pulse wave measurement, and recording measurement results and the like. The display section 150 is configured, for example, by an LCD and the like, and is means for displaying the measurement results and the like. The operation section 160 is means for accepting the operation by the subject and the like, and inputting such a command from the outside to the CPU 130 and the power supply section 170. The power supply section 170 is means for supplying power serving as a power source to the CPU 130.

The CPU 130 inputs a control signal for driving the constant current supply section 110 to the constant current supply section 110, and inputs volume pulse wave information serving as the measurement result to the memory section 140 and the display section 150. The CPU 130 also includes a volume pulse wave acquiring unit 131 for acquiring a volume pulse wave, and the volume pulse wave acquiring unit 131 acquires the volume pulse wave of the radial artery based on the fluctuation information of the biological impedance measured by the impedance measurement section 120. The volume pulse wave information acquired by the volume pulse wave acquiring unit 131 is inputted to the memory section 140 and the display section 150 as a measurement result.

The pulse wave measurement device 100A may separately include an output section for outputting the volume pulse wave information serving as a measurement result to an external device and the like (e.g., biological information measurement device such as sphygmomanometer). A serial communication circuit, a write device to various types of recording medium, and the like can be used for the output section. According to such a configuration, the volume pulse wave information can be directly or indirectly outputted to the external device and the like.

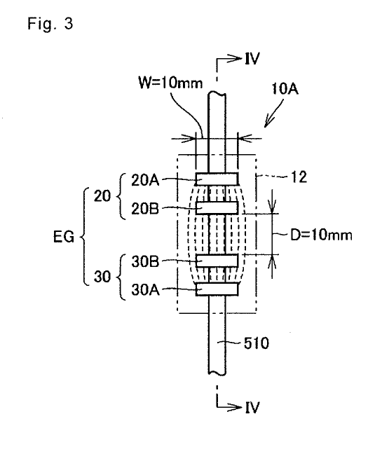

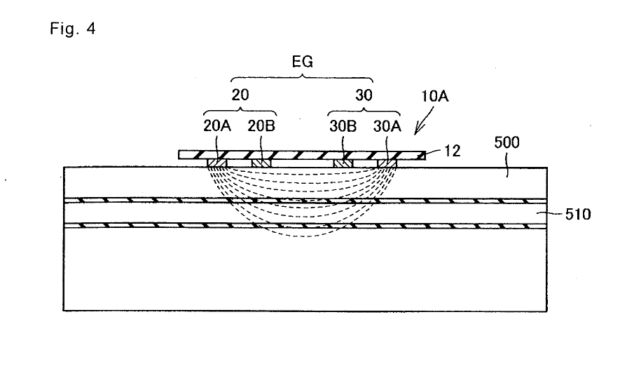

FIGS. 3 and 4 are views showing a state in which the pulse wave measurement electrode unit is attached to the wrist in the pulse wave measurement device according to the present embodiment, where FIG. 3 is a plan view in the attached state and FIG. 4 is a schematic cross-sectional view taken along line IV-IV shown in FIG. 3. The state in which the pulse wave measurement electrode unit 10A according to the present embodiment is attached to the wrist will now be described with reference to FIGS. 3 and 4.

As shown in FIGS. 3 and 4, the contacting surfaces 20As, 20Bs, 30As, and 30Bs of the electrodes 20A, 20B, 30A, and 30B are brought into contact with a surface of a wrist 500 when the pulse wave measurement electrode unit 10A according to the present embodiment is attached to the wrist 500. Since the electrodes 20A, 20B, 30A, and 30B are arranged aligned in a line on the supporting member 12, an extending direction of a radial artery 510 substantially coincides the aligning direction of the electrodes 20A, 20B, 30A, and 30B by positioning and arranging the electrodes 20A, 20B, 30A, and 30B on a skin of the wrist 500 at a portion where the radial artery 510 extends when attaching the pulse wave measurement electrode unit 10A to the wrist 500.

A constant current is supplied between the pair of current application electrodes 20A, 30A by the constant current supply section 110 in this state, and the potential difference created at this time between the pair of voltage measurement electrodes 20B, 30B is measured by the impedance measurement section 120 to measure the biological impedance at the measuring site. The biological impedance obtained in this manner and time are associated, so that the fluctuation of the biological impedance is detected, and the volume pulse wave of the radial artery 510 is acquired in the volume pulse wave acquiring unit 131 based on such information. A current path in the wrist 500 is schematically shown with a broken line in FIGS. 3 and 4. As shown in the figure, the current path that forms in the wrist 500 during measurement has a constant spreading in a direction orthogonal to the extending direction of the radial artery 510 (i.e., aligning direction of electrodes 20A, 20B, 30A, and 30B) and a depth direction, and is formed towards a direction parallel to the extending direction of the radial artery 510.

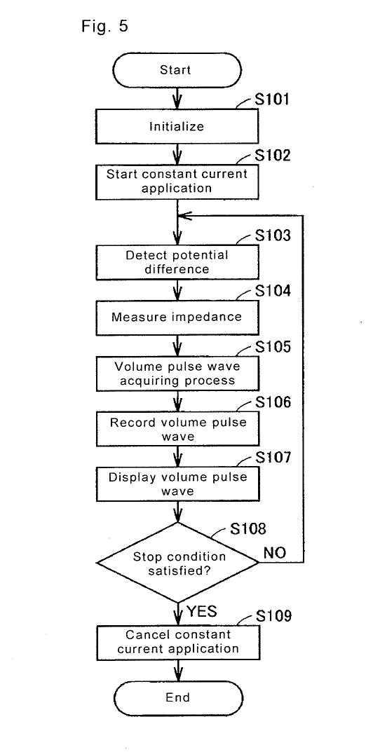

FIG. 5 is a flowchart showing a processing procedure of the pulse wave measurement device according to the present embodiment. The processing procedure in the pulse wave measurement device 100A according to the present embodiment will be described with reference to FIG. 5. The program complying with the flowchart is stored in advance in the memory section 140 shown in FIG. 1, and the process proceeds by having the CPU 130 read out and execute the program from the memory section 140.

As shown in FIG. 5, when the subject operates the operation section 160 of the pulse wave measurement device 100A to input the command of power ON, power serving as a power supply is supplied from the power supply section 170 to the CPU 130 thereby driving the CPU 130, and initialization of the pulse wave measurement device 100A is performed (step S101). The subject positions and attaches in advance the pulse wave measurement electrode unit 10A described above at a predetermined position of the wrist 500.

The subject then operates an operation button of the operation section 160 of the pulse wave measurement device 100A to input the command to start measurement, whereby the CPU 130 gives an instruction to start constant current application to the constant current supply section 110. The constant current supply section 110 then supplies a constant current between the pair of current application electrodes 20A, 30A (step S102). Thereafter, the CPU 130 gives an instruction to detect the potential difference to the impedance measurement section 120. The potential difference between the pair of voltage measurement electrodes 20B, 30B is detected in the impedance measurement section 120 (step S103), and the biological impedance is measured (step S104). The detected biological impedance is then digitized by the impedance measurement section 120 and inputted to the CPU 130, and the volume pulse wave is acquired in the volume pulse wave acquiring unit 131 (step S105). The acquired volume pulse wave is stored in the memory section 140 as a measurement result (step S106), and then displayed on the display section 150 (step S107). The display section 150 displays the volume pulse wave, for example, as numerical values or waveforms.

The series of operations from step S103 to step S107 are repeatedly performed (when NO in step S108) until a predetermined stop condition (e.g., operation of measurement stop switch by user, elapse of set time by timer circuit, and the like) is satisfied. When the predetermined stop condition is satisfied (when YES in step S108), the CPU 130 gives an instruction to cancel the constant current application to the constant current supply section 110 (step S109). The pulse wave measurement device 100A then takes a standby state, and the supply of power serving as a power supply is stopped after input of command of power OFF by the operation section 160 of the subject is made. Therefore, the volume pulse wave that changes with time can be measured in real time.

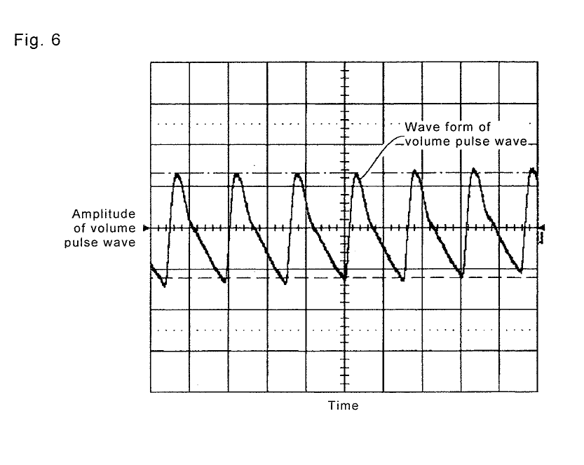

FIG. 6 is a graph showing the waveform of a volume pulse wave actually obtained by the pulse wave measurement device 100A according to the present embodiment. In FIG. 6, a horizontal axis indicates time and a vertical axis indicates amplitude of the volume pulse wave.

The waveform of the volume pulse wave shown in FIG. 6 is obtained when the electrode layout shown in FIG. 3 is adopted. In other words, as shown in FIG. 3, a width (electrode width) W of each electrode 20A, 20B, 30A, 30B is 10 mm, and a distance (inter-electrode portion distance) D between the first electrode portion 20 and the second electrode portion 30 is 10 mm. The electrode width W is the length of each electrode in the direction orthogonal to the extending direction of the radial artery 510 when the pulse wave measurement electrode unit 10A is attached to the wrist 500. When such an electrode layout is adopted, the waveform of the volume pulse wave can be measured at satisfactory precision, as shown in FIG. 6. In the pulse wave measurement device 100A according to the present embodiment, the waveform of the volume pulse wave shown in FIG. 6 is displayed by the display section 150.

As described above, in the pulse wave measurement electrode unit 10A and the pulse wave measurement device 100A equipped with the same according to the present embodiment, the pair of current application electrodes 20A, 30A and the pair of voltage measurement electrodes 20B, 30B are linearly arranged, and each electrode 20A, 20B, 30A, 30B is supported by the supporting member 12 such that the aligning direction of the electrodes 20A, 20B, 30A, and 30B substantially coincide the extending direction of the radial artery 510 when the pulse wave measurement electrode unit 10A is attached to the wrist 500, and thus a body tissue portion other than the radial artery 510 is excluded as much as possible from being included at the measuring site (i.e., measuring site or site where the applied constant current passes) positioned between the first electrode portion 20 and the second electrode portion 30. Thus, the impedance fluctuation at the body tissue portion other than the radial artery 510 is suppressed from being superimposed on the measured volume pulse wave as an error component of the volume pulse wave measurement, and the pulse wave measurement device capable of measuring the volume pulse wave at high precision compared to the related art and the pulse wave measurement electrode unit used therefor can be obtained.

In the pulse wave measurement electrode unit 10A and the pulse wave measurement device 100A equipped with the same according to the present embodiment, the contacting surfaces 20Aₛ, 20Bₛ, 30Aₛ, and 30Bₛ of the pair of current application electrodes 20A, 30A and the pair of voltage measurement electrodes 20B, 30B with respect to the wrist 500 are positioned on an identical plane, and thus the contacting state of the electrodes 20A, 20B, 30A, and 30B with respect to the wrist 500 can be stabilized, and the fluctuation in contact resistance during the measurement can be suppressed. Therefore, high precision volume pulse wave measurement can be realized from this aspect as well.

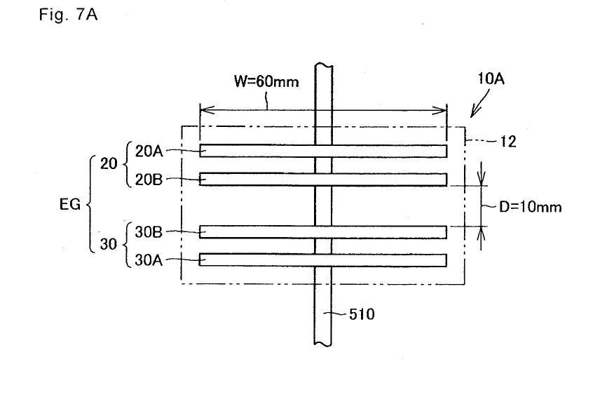

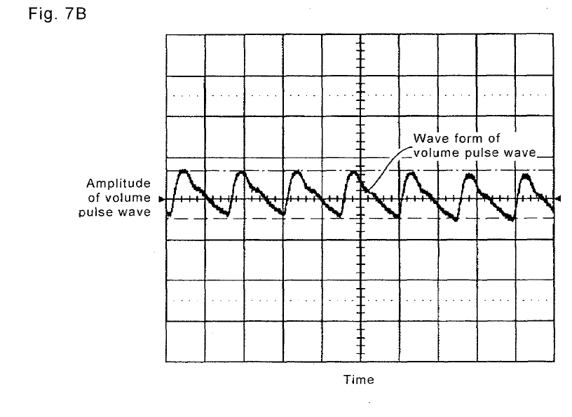

Examples where the electrode layout of the pulse wave measurement electrode unit is variously changed in the pulse wave measurement device according to the present embodiment will be described, and the suitable electrode layout will be described based on the examples. FIGS. 7A, 8A, 9A, and 10A are electrode layout diagrams each showing an example where the electrode layout of the pulse wave measurement electrode unit is variously changed in the pulse wave measurement device according to the present embodiment. FIGS. 7B, 8B, 9B and 10B are graphs respectively showing the waveform of the volume pulse wave obtained when the electrode layout shown in FIGS. 7A, 8A, 9A, and 10A is adopted.

The electrode layout shown in FIG. 7A is a case where the electrode width W of each electrode 20A, 20B, 30A, 30B is 60 mm, and the inter-electrode portion distance D between the first electrode portion 20 and the second electrode portion 30 is 10 mm. Compared to when the electrode layout shown in FIG. 3 is adopted, the amplitude of the waveform of the measured volume pulse wave is reduced as shown in FIG. 7B when such an electrode layout is adopted. This is assumed to be because more body tissue portion other than the radial artery 510 is included in the measuring site or the site where the applied constant current passes with increase in the electrode width W. Therefore, determination is made that the width of between about 5 mm and 15 mm, slightly larger than the diameter (normally, between about 1.2 mm and 3.5 mm) of the radial artery is particularly suitable for the electrode width W of each electrode 20A, 20B, 30A, and 30B.

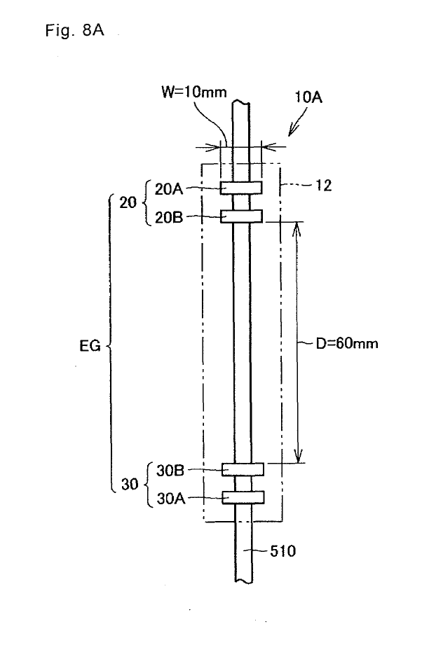

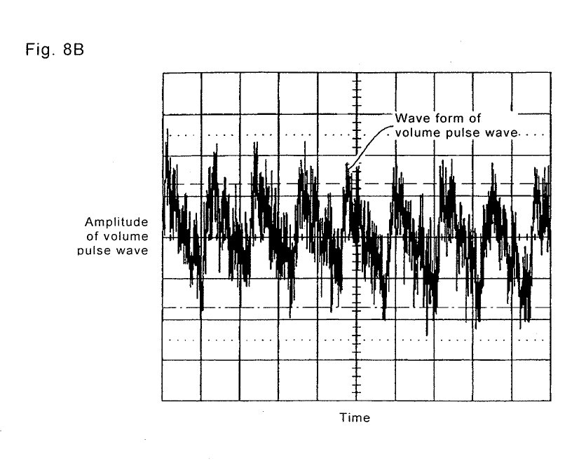

The electrode layout shown in FIG. 8A is a case where the electrode width W of each electrode 20A, 20B, 30A, 30B is 10 mm, and the inter-electrode portion distance D between the first electrode portion 20 and the second electrode portion 30 is 60 mm. Compared to when the electrode layout shown in FIG. 3 is adopted, the amplitude of the waveform of the measured volume pulse wave is not reduced but a very large disturbance is produced at the waveform as shown in FIG. 8B when such an electrode layout is adopted. This is assumed to be because when the inter-electrode portion distance D is increased on the wrist 500 attached with the pulse wave measurement electrode unit 10A, if one electrode portion is arranged on the wrist 500, the other electrode portion is arranged at a position on the elbow side than the wrist 500. In other words, the radial artery 510 runs through a relatively shallow position under the skin at the wrist 500 but runs through a deeper position under the skin towards the elbow side, and thus more body tissue portion other than the radial artery 510 is included at the measuring site or the site where the applied constant current passes. Therefore, determination is made that the inter-electrode portion distance D between the first electrode portion 20 and the second electrode portion 30 is preferably, in particular, between about 10 mm and 20 mm. However, the setting of the inter-electrode portion distance D that takes into consideration that a sufficiently stable constant current can be supplied to the radial artery 510 positioned under the skin by the current application electrodes 20A, 30A between the voltage measurement electrodes 20B, 30B, and that a sufficient potential difference can be detected between the voltage measurement electrodes 20B, 30B is required.

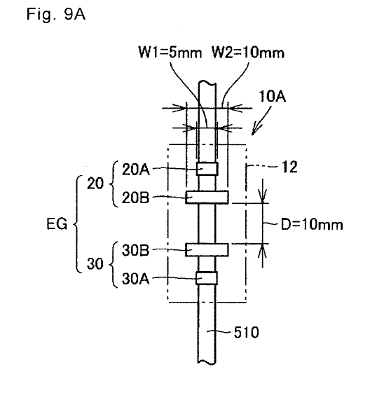

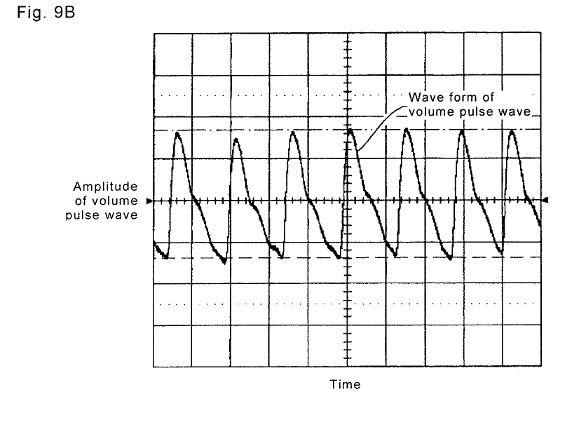

The electrode layout shown in FIG. 9A is a case where an electrode width W1 of the pair of current application electrodes 20A, 30A is 5.0 mm, an electrode width W2 of the pair of voltage measurement electrodes 20B, 30B is 10 mm, and the inter-electrode portion distance D between the first electrode portion 20 and the second electrode portion 30 is 10 mm. Compared to when the electrode layout shown in FIG. 3 is adopted, the waveform of the measured volume pulse wave can be obtained at higher precision as shown in FIG. 9B when such an electrode layout is adopted.

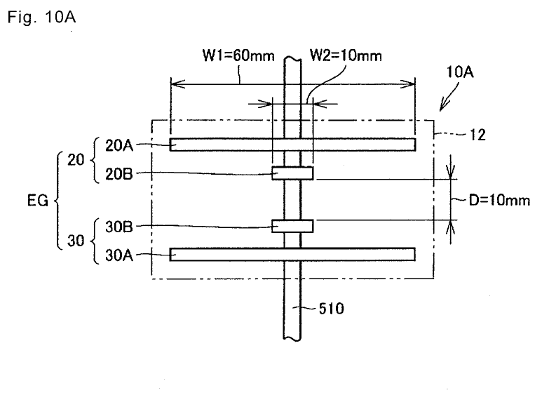

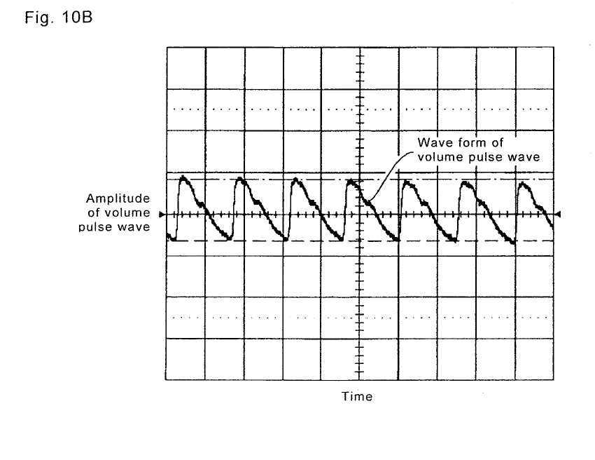

On the other hand, the electrode layout shown in FIG. 10A is a case where the electrode width W1 of the pair of current application electrodes 20A, 30A is 60 mm, the electrode width W2 of the pair of voltage measurement electrodes 20B, 30B is 10 mm, and the inter-electrode portion distance D between the first electrode portion 20 and the second electrode portion 30 is 10 mm. Compared to when the electrode layout shown in FIG. 3 is adopted, the amplitude of the waveform of the measured volume pulse wave is reduced as shown in FIG. 10B when such an electrode layout is adopted.

Comparing the electrode layout shown in FIG. 9A and the electrode layout shown in FIG. 10A, the volume pulse wave measurement of high precision can be performed with the electrode layout shown in FIG. 9 with which the body tissue portion other than the radial artery 510 is not included in great amount at the measuring site or the site where the applied constant current passes and the constant current can be locally applied than with the electrode layout shown in FIG. 10A where a constant current is applied over a wide region. Therefore, high precision volume pulse wave measurement can be performed if the length (electrode width W1) of the current application electrodes 20A, 30A in the direction intersecting the aligning direction of the electrodes 20A, 20B, 30A, and 30B (i.e., direction in which the first electrode portion 20 and the second electrode portion 20 line) is equal to or less than the length (electrode width W2) of the voltage application measurement electrodes 20B, 30B in the direction intersecting the aligning direction of the electrodes 20A, 20B, 30A, and 30B (i.e., direction in which the first electrode portion 20 and the second electrode portion 20 line).

### Second Embodiment

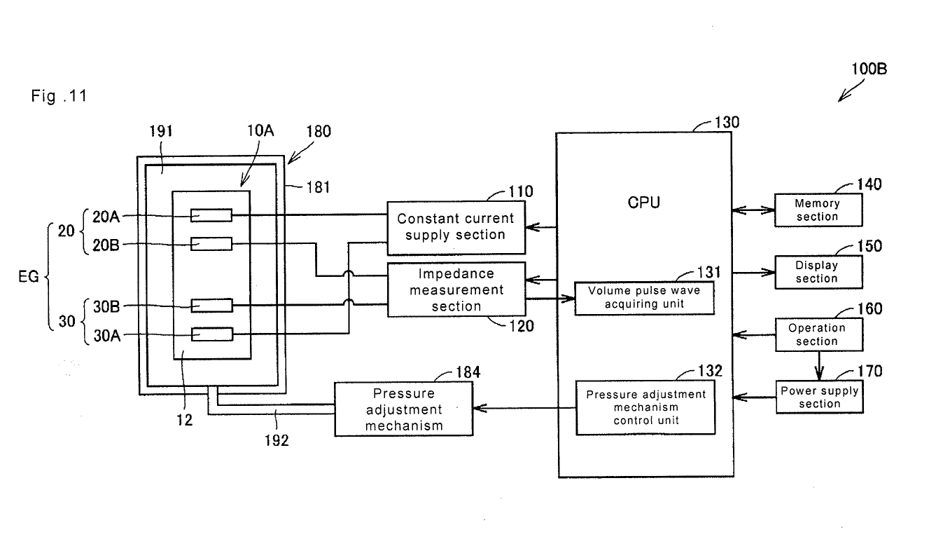

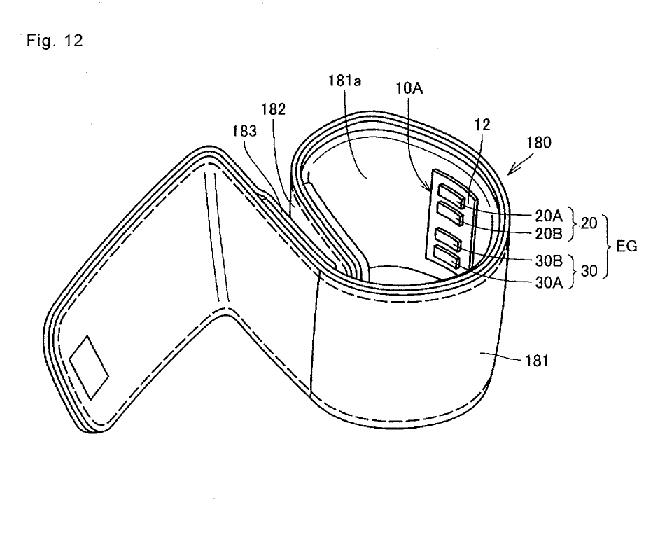

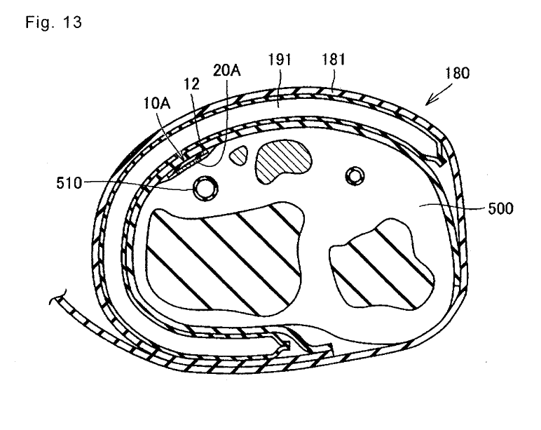

FIG. 11 is a function block diagram showing a configuration of a pulse wave measurement device according to a second embodiment of the present invention, and FIG. 12 is a schematic perspective view of a cuff of the pulse wave measurement device according to the present embodiment. FIG. 13 is a cross-sectional view showing a state in which the cuff of the pulse wave measurement device according to the present embodiment is attached to the wrist. A configuration of a pulse wave measurement device 1008 according to the present embodiment and a structure of a cuff 180 will be described with reference to FIGS. 11 to 13. In the figure, the same reference symbols are denoted for the portions similar to the pulse wave measurement device 100A according to the first embodiment, and the description thereof will not be repeated.

As shown in FIGS. 11 to 13, the pulse wave measurement device 100B according to the present embodiment is equipped with a compression mechanism capable of lightly compressing the radial artery 510. The compression mechanism is configured by an air bag 191 provided in the cuff 180 to be wrapped around the wrist 500, and a pressure adjustment mechanism 184 for adjusting an inner pressure (hereinafter also referred to as cuff pressure) of the air bag 191.

More specifically, the air bag 191 is a bag-shaped member made of rubber or resin that freely expands and contracts by injecting air thereto or discharging the injected air to the outside. The air bag 191 is included in a cuff cover 181 made of cloth, where the air bag 191 and the cuff cover 181 constitute the cuff 180. The air bag 191 is fixed to the wrist 500 by wrapping the cuff 180 around the wrist 500. As the air bag 191 expands with the cuff 180 attached to the wrist 500, the radial artery 510 is lightly compressed by the air bag 191. In this case, the inner peripheral surface of the air bag 191 functions as a compression acting surface.

As shown in FIGS. 12 and 13, the pulse wave measurement electrode unit 10A is attached to a predetermined position on an inner peripheral surface 181a of the cuff 180. The air bag 191 is positioned in the interior of the portion attached with the pulse wave measurement electrode unit 10A of the cuff 180. Thus, the pulse wave measurement electrode unit 10A is positioned on the inner peripheral surface or the compression acting surface of the air bag 191. Surface fasteners 182, 183 (see FIG. 12) serving as fixing members for maintaining the attached state of the cuff 180 to the wrist 500 are provided at predetermined positions of the cuff cover 181.

As shown in FIG. 11, the pressure adjustment mechanism 184 is connected to the air bag 191 by way of an air tube 192. The pressure adjustment mechanism 184 is configured by a pump, a valve, and the like, which operation is controlled by a pressure adjustment mechanism control unit 132 arranged in the CPU 130.

As shown in FIGS. 11 and 12, the pulse wave measurement electrode unit 10A has a similar configuration as that of the first embodiment described above, and is attached on the inner peripheral surface 181a of the cuff 180 such that the aligning direction of the pair of current application electrodes 20A, 30A and the pair of voltage measurement electrodes 20B, 30B is parallel to the axial direction of the cuff 180 wrapped around the wrist 500 in a substantially cylindrical shape. Therefore, when the cuff 180 is attached to the wrist 500, the extending direction of the radial artery 510 and the aligning direction of the electrodes 20A, 20B, 30A, and 30B substantially coincide, and the electrodes 20A, 20B, 30A, and 30B are brought into contact with the surface of the wrist 500. The pulse wave measurement electrode unit 10A is pressed against the surface of the wrist 500 when the air bag 191 arranged in the cuff 180 is expanded by the pressure adjustment mechanism 184. The supporting member 12 for supporting the electrodes 20A, 20B, 30A, and 30B may be configured by a film-shaped resin member having poor rigidity, or may be configured by a hard resin member and the like having an appropriate rigidity. If the compression mechanism is arranged in the pulse wave measurement electrode unit 10A as in the present embodiment, the supporting member 12 itself including the resin member may be omitted, and the electrodes 20A, 20B, 30A, and 30B may be directly attached to the inner peripheral surface 181a of the cuff 180. In this case, the cuff 180 serves as the supporting member for supporting the electrodes 20A, 20B, 30A, and 30B.

With the pulse wave measurement device 100B having such a configuration, the pulse wave measurement electrode unit 10A can be pressed towards the wrist 500 while lightly compressing the radial artery 510. Therefore, the contact stability of the electrodes 20A, 20B, 30A, and 30B with respect to the wrist 500 can be ensured, and the radial artery 510 is moderately lightly compressed, so that high precision pulse wave measurement can be performed. A magnitude of the compression force with respect to the wrist 500 using the compression mechanism is preferably a compression force to an extent the compression force of about an average blood pressure value of the subject exerts on the radial artery 510. According to such a configuration, the volume pulse wave can be measured in a state where the amplitude becomes a maximum.

In order to measure the volume pulse wave in a state where the amplitude becomes a maximum, the compression force exerted on the radial artery 510 is monitored, and the pressure adjustment mechanism 184 needs to be controlled by the pressure adjustment mechanism control unit 132 such that the relevant compression force becomes about the average blood pressure value of the subject. However, it is not possible to directly monitor the compression force on the radial artery 510, and thus in order to measure the volume pulse wave in a state where the amplitude becomes a maximum, the inner pressure of the air bag 191 is monitored using a pressure sensor and the like assuming the inner pressure of the air bag 191 is equal to the compression force exerted on the radial artery 510, and the pressure adjustment mechanism 184 is controlled by the pressure adjustment mechanism control unit 132 such that the inner pressure of the air bag 191 becomes about the average blood pressure value of the subject.

However, in the case of the pulse wave measurement device 100B having the above configuration, the electrodes 20A, 20B, 30A, and 30B exist between the air bag 191 and the wrist 500 as obstacles, and thus the compression force actually exerted on the radial artery 510 may not be equal to the inner pressure of the air bag 191 even if the inner pressure of the air bag 191 is set to about the average blood pressure value of the subject. In such a state, the volume pulse wave cannot be measured in a state where the amplitude becomes a maximum, and becomes a cause of inhibition of the high precision volume pulse wave measurement. The configuration of the pulse wave measurement device that can solve such a problem will be described below.

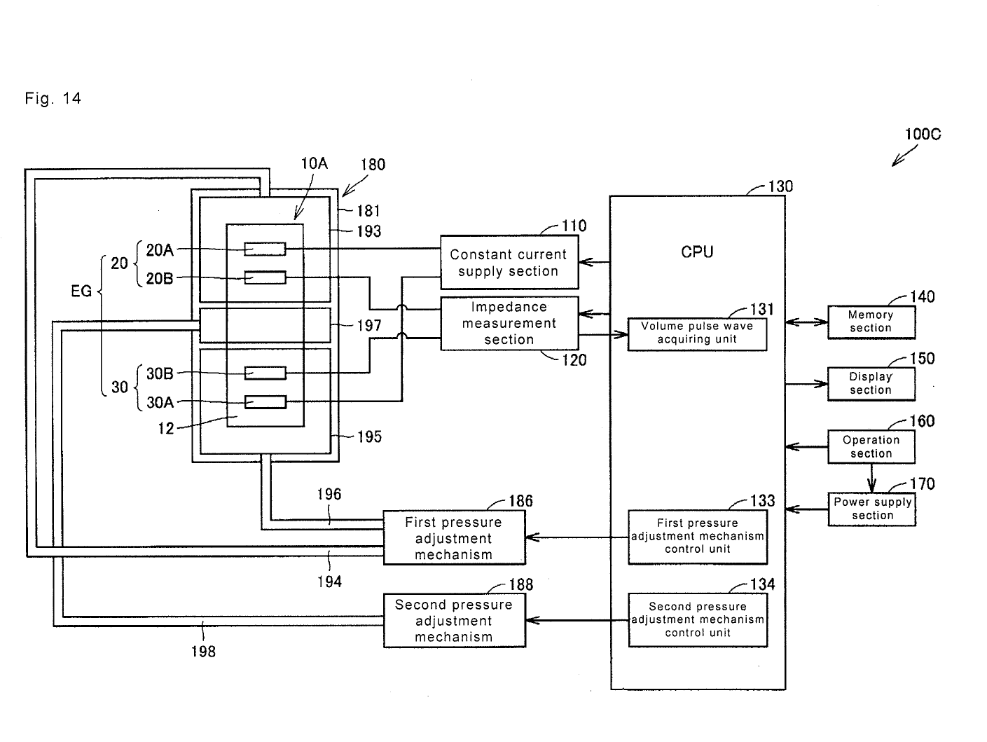

FIG. 14 is a function block diagram showing another configuration example of the pulse wave measurement device according to the present embodiment. A pulse wave measurement device 100C according to the present configuration example will be described below with reference to FIG. 14. In the figure, the same reference symbols are denoted for the portions similar to the pulse wave measurement device 100B according to the present embodiment, and the description thereof will not be repeated.

As shown in FIG. 14, in the pulse wave measurement device 100C according to the present configuration example, the air bag arranged in the cuff 180 is divided into three, a first air bag 193, a second air bag 195, and a third air bag 197, where the first air bag 193 is arranged at a position corresponding to the first electrode portion 20 of the pulse wave measurement electrode unit 10A, the second air bag 195 is arranged at a position corresponding to the second electrode portion 30 of the pulse wave measurement electrode unit 10A, and the third air bag 197 is arranged at a position corresponding to the portion between the first electrode portion 20 and the second electrode portion 30 of the pulse wave measurement electrode unit 10A. The first air bag 193 and the second air bag 195 are connected to a first pressure adjustment mechanism 186 by way of air tubes 194, 196, respectively, and the third air bag 197 is connected to a second pressure adjustment mechanism 188 by way of an air tube 198. An operation of the first pressure adjustment mechanism 186 is controlled by a first pressure adjustment mechanism control unit 133 arranged in the CPU 130, and an operation of the second pressure adjustment mechanism 188 is controlled by a second pressure adjustment mechanism control unit 134 arranged in the CPU 130.

In other words, in the pulse wave measurement device 100C according to the present configuration example, the portion positioned with the first electrode portion 20 and the second electrode portion 30 of the supporting member 12 of the pulse wave measurement electrode unit 10A is pressed towards the wrist 500 with a first compression mechanism including the first air bag 193, the second air bag 195, and the first pressure adjustment mechanism 186, and the portion positioned with the first electrode portion 20 and the second electrode portion 30 of the supporting member 12 of the pulse wave measurement electrode unit 10A is pressed towards the wrist 500 with a second compression mechanism including the third air bag 197 and the second pressure adjustment mechanism 188.

According to such a configuration, the portion positioned with the electrodes 20A, 20B, 30A, and 30B serving as obstacles and the portion not positioned therewith of the supporting member 12 of the pulse wave measurement electrode unit 10A can be pressed against the wrist 500 separately and independently from each other with different compression mechanisms. The portion positioned with the electrodes 20A, 20B, 30A, and 30B serving as obstacles of the supporting member 12 is pressed by the first compression mechanism for pressing the relevant portion at a pressure less than the average blood pressure value of the subject, and the portion not positioned with the electrodes 20A, 20B, 30A, and 30B serving as obstacles of the supporting member 12 is pressed by the second compression mechanism for pressing the relevant portion at a pressure of about the average blood pressure value of the subject, so that the measurement of the volume pulse wave can be more reliably carried out in a state where the amplitude becomes a maximum. Therefore, the volume pulse wave measurement of higher precision can be realized.

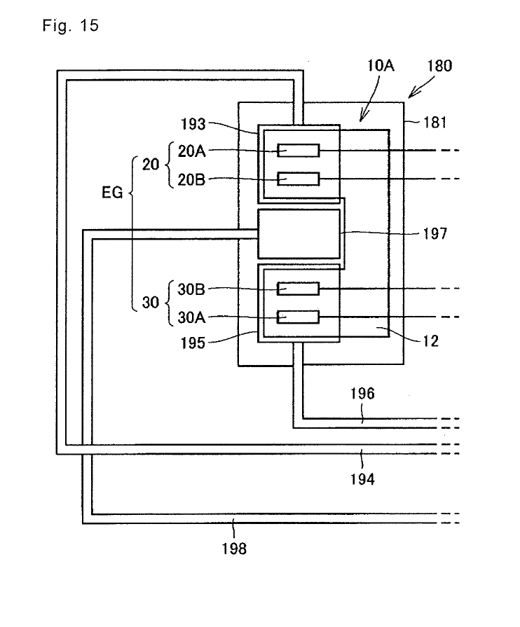

In the pulse wave measurement device 100C according to the above configuration example, the volume pulse wave measurement can be performed at higher precision by adopting the configuration shown in FIG. 15. In the configuration shown in FIG. 15, the supporting member 12 extends so as to circumvent the portion positioned between the first electrode portion 20 and the second electrode portion 30. According to such a configuration, the wrist 500 can be directly compressed without having the supporting member 12 by the third air bag 197 arranged in correspondence to the portion positioned between the first electrode portion 20 and the second electrode portion 30, and the control of the pressure adjustment mechanism is facilitated.

In the pulse wave measurement devices 1008, 100C described in the present embodiment, a case of adopting the air bag for the compression mechanism to lightly compress the radial artery has been illustrated and described, but it should be recognized that other means can also be used. For instance, a fluid bag to which other gas or a fluid such as a liquid is injected instead of air may be used, or the supporting member may be pressed against the wrist using an actuator typified by a motor or the like.

### Third Embodiment

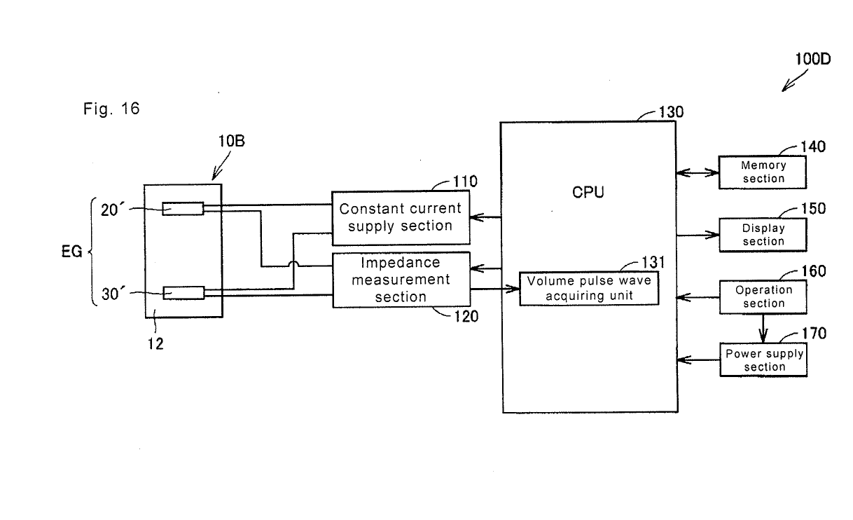

FIG. 16 is a function block diagram showing a configuration of a pulse wave measurement device according to a third embodiment of the present invention. A configuration of a pulse wave measurement device 100D according to the present embodiment will be described with reference to FIG. 16. In the figure, the same reference symbols are denoted for the portions similar to the pulse wave measurement device 100A according to the first embodiment, and the description thereof will not be repeated.

As shown in FIG. 16, the pulse wave measurement device 100D according to the present embodiment differs from the pulse wave measurement device 100A according to the first embodiment in the configuration of the pulse wave measurement electrode unit. In other words, in the pulse wave measurement electrode unit 10B of the pulse wave measurement device 100D according to the present embodiment, one of the pair of current application electrodes and one of the pair of voltage measurement electrodes are both used by a single electrode 20′, and the other one of the pair of current application electrodes and the other one of the pair of voltage measurement electrodes are both used by a single electrode 30′. In other words, only two electrodes, first current application and voltage measurement electrode 20′ and second current application and voltage measurement electrode 30′, are arranged on the main surface of the supporting member 12.

Even with such a configuration, the volume pulse wave can be measured as the electrodes 20′, 30′ are supported by the supporting member 12 such that the pair of current application and voltage measurement electrodes 20′, 30′ are arranged lined in the extending direction of the radial artery while the pulse wave measurement electrode unit 10B is attached to the wrist. According to such a configuration, the pulse wave measurement electrode unit can be realized with a simpler configuration.

### Fourth Embodiment

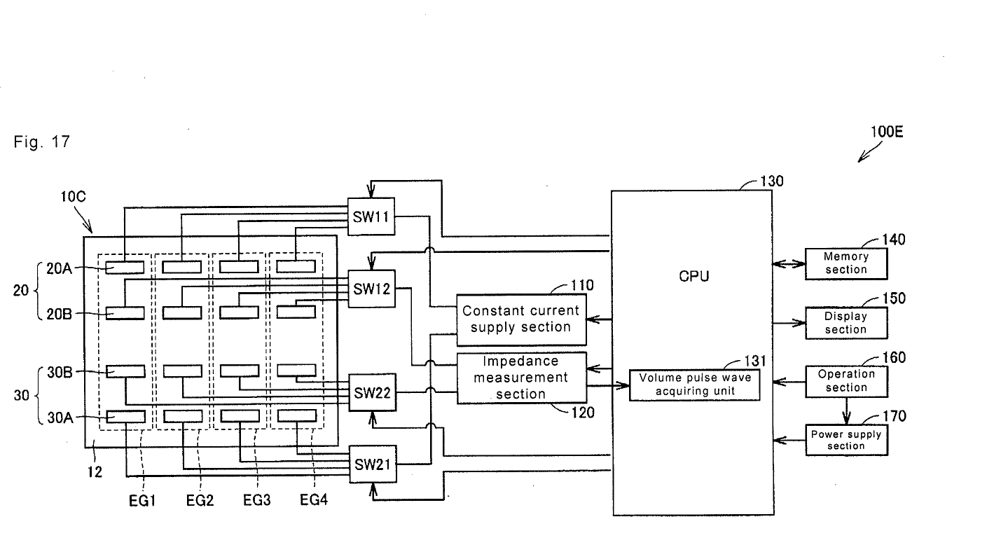

FIG. 17 is a function block diagram showing a configuration of a pulse wave measurement device according to a fourth embodiment of the present invention. A configuration of a pulse wave measurement device 100E according to the present embodiment will be described below with reference to FIG. 17. In the figure, the same reference symbols are denoted for the portions similar to the pulse wave measurement device 100A according to the first embodiment, and the description thereof will not be repeated.

In the first to the third embodiments, the pulse wave measurement devices 100A to 100D for measuring the volume pulse wave using the pulse wave measurement electrode units 10A, 10B arranged with a set of electrode groups have been described. However, when adopting such a configuration, two or four electrodes in the electrode group need to be accurately positioned and brought into contact with the surface of the skin located above the radial artery of the wrist, and a very strict positioning task is required. A pulse wave measurement electrode unit 10C and the pulse wave measurement device 100E equipped with the same according to the present embodiment do not require such a strict positioning task.

As shown in FIG. 17, the pulse wave measurement electrode unit 10C of the pulse wave measurement device 100E according to the present embodiment includes a plurality of sets of electrode groups including the electrodes 20A, 20B, 30A, and 30B. Specifically, as shown in the figure, four sets of electrode groups, or first to fourth electrode groups EG1 to EG4, are arranged, and the electrodes arranged on the main surface of the supporting member 12 are in a total of sixteen, four vertically×four horizontally. Such electrodes are arranged in an array form.

Each of the first to the fourth electrode groups EG1 to EG4 includes the first electrode portion 20 and the second electrode portion 30 arranged with a predetermined distance from the first electrode portion 20, similar to the pulse wave measurement electrode unit 10A in the first embodiment. Each of the first electrode portion 20 includes two electrodes that are separated and independent, and includes a first current application electrode 20A, which is one of the pair of current application electrodes, and a first voltage measurement electrode 20B, which is one of the pair of voltage measurement electrodes. Each of the second electrode portion 30 includes two electrodes that are separated and independent, and includes a second current application electrode 30A, which is the other of the pair of current application electrodes, and a second voltage measurement electrode 30B, which is the other of the pair of voltage measurement electrodes.

Each of the electrodes 20A, 20B, 30A, and 30B included in each of the first to the fourth electrode groups EG1 to EG4 is formed in a substantially rectangular shape in plan view as shown in the figure. The pair of voltage measurement electrodes 20B, 30B are sandwiched by the pair of current application electrodes 20A, 30A, so that the electrodes 20A, 20B, 30A, and 30B included in each of the first to the fourth electrode groups EG1 to EG4 are arranged aligned in a line on the supporting member 12. The supporting member 12 supports the first to the fourth electrode groups EG1 to EG4 so that the aligning direction of the electrodes 20A, 20B, 30A, and 30B included in each of the first to the fourth electrode groups EG1 to EG4 coincides with the extending direction of the radial artery extending through the wrist when the pulse wave measurement electrode unit 10C is attached to the wrist. In other words, the supporting member 12 supports the first to the fourth electrode groups EG1 to EG4 such that the first to the fourth electrode groups EG1 to EG4 are arranged in a line in a direction intersecting the direction in which the first electrode portion 20 and the second electrode portion 30 are lined. The main surfaces of the total of sixteen electrodes does not necessarily all need to be positioned on an identical plane, and the main surfaces of four electrodes included in each of the first to the fourth electrode groups EG1 to EG4 merely need to be positioned on the identical plane.

As shown in FIG. 17, the pulse wave measurement device 100E according to the present embodiment includes switches SW11, SW12 serving as a first electrode portion selecting unit for switchably selecting a specific first electrode portion of the four first electrode portions 20 included in the pulse wave measurement electrode unit 10C, and switches SW21, SW22 serving as a second electrode portion selecting unit for switchably selecting a specific second electrode portion of the four second electrode portions 30 included in the pulse wave measurement electrode unit 10C. The operation of each of the switches SW11, SW12, SW21, and SW22 is controlled by the CPU 13, and only the first electrode portion and the second electrode portion selected by the switches SW11, SW12, SW21, and SW22 are electrically connected to the constant current supply section 110 and the impedance measurement section 120.

The volume pulse wave can be measured by switching the switches SW11, SW12, SW21, and SW22 and selecting each of the first to the fourth electrode groups EG1 to EG4 by adopting the above-described configuration, and the information from which the amplitude of the volume pulse wave is obtained the largest of the obtained volume pulse wave information can be used as the measurement result. Therefore, strict positioning on the wrist of the pulse wave measurement electrode unit 10C is not required, and the positioning task can be facilitated. The pulse wave measurement electrode unit and the pulse wave measurement device excelling in convenience are thus obtained.

In the impedance measurement using the selected electrode group, the impedance measurement using one of the non-selected electrode group (preferably, electrode group at a position most distant from the selected electrode group) may be performed at the same time so that the impedance fluctuation measured by the non-selected electrode group can be handled as a reference potential fluctuation of the living body, which may be subtracted from the impedance fluctuation measured by the selected electrode group to measure the volume pulse wave at higher precision.

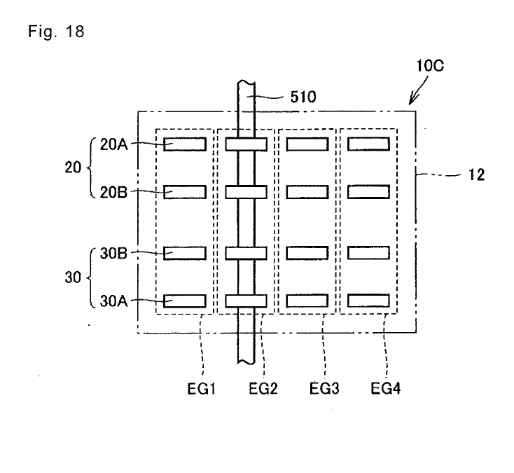

FIGS. 18 to 20 are views each variously showing a positional relationship between the electrode and the radial artery when the pulse wave measurement electrode unit is attached to the wrist in the pulse wave measurement device according to the present embodiment. In the pulse wave measurement device 100E according to the present embodiment, a case of switching the switches SW11, SW12, SW21, and SW22 to select each of the first to the fourth electrode groups EG1 to EG4, and measuring the volume pulse wave has been illustrated and described. Such a configuration exhibits effects when the aligning direction of the four electrodes 20A, 20B, 30A, and 30B included in each of the first to the fourth electrode groups EG1 to EG4 is substantially parallel to the extending direction of the radial artery 510 and the radial artery 510 is positioned below one of the first to the fourth electrode groups EG1 to EG4 when the pulse wave measurement electrode unit 10C is attached to the wrist, as shown in FIG. 18.

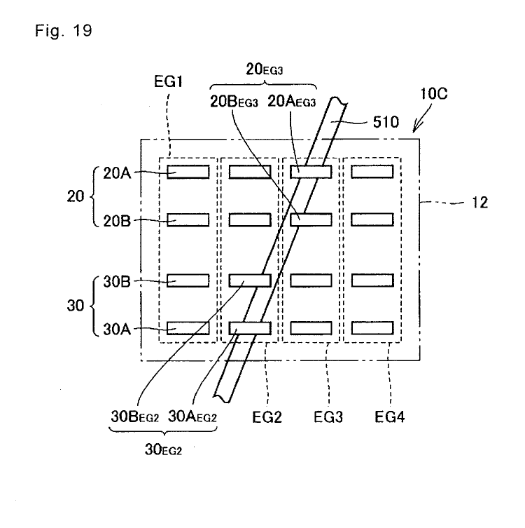

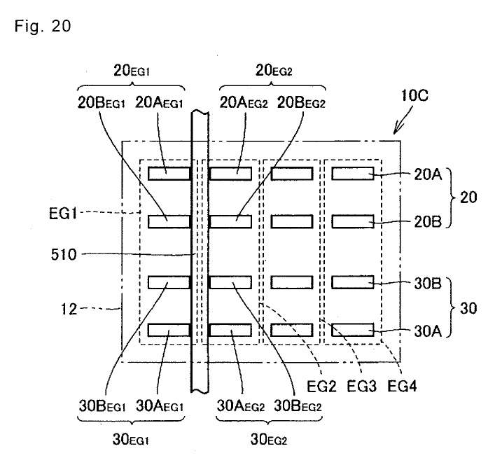

However, when the aligning direction of the electrodes 20A, 20B, 30A, and 30B and the extending direction of the radial artery 510 are inclined with an angle of a certain extent when the pulse wave measurement electrode unit 10C is attached to the wrist, as shown in FIG. 19, or when the radial artery 510 is positioned in a gap portion between the first to the fourth electrode groups EG1 to EG4, as shown in FIG. 20, the volume pulse wave measurement of high precision may not necessarily be possible. However, even in such cases, the volume pulse wave can be measured by further variously changing the switching of the switches SW11, SW12, SW21, and SW22, and thus a degree of freedom in the attachment position of the pulse wave measurement electrode unit during measurement increases with the pulse wave measurement device according to the present embodiment. The switching examples will be described below.

First, in the case shown in FIG. 19, a first current application electrode 20A_{EG3} and a first voltage measurement electrode 20B_{EG3} of a first electrode portion 20_{EG3} of the third electrode group EG3 are respectively connected to the constant current supply section 110 and the impedance measurement section 120 as the specific first electrode portion by switching the switches SW11, SW12. A second current application electrode 30A_{EG2} and a second voltage measurement electrode 30B_{EG2} of a second electrode portion 30_{EG2} of the second electrode group EG2 are respectively connected to the constant current supply section 110 and the impedance measurement section 120 as the specific second electrode portion by switching the switches SW21, SW22. Thus, high precision volume pulse wave measurement can be performed by selecting the first electrode portion and the second electrode portion closest to the skin located immediately above the radial artery 510 as the pulse wave measurement electrodes and performing the measurement of the volume pulse wave.

The selection of the first electrode portion and the second electrode portion by the switches SW11, SW12, SW21, and SW22 is not necessarily limited to simultaneously selecting the first electrode portion and the second electrode portion included in a single electrode group, and the first electrode portion and the second electrode portion between different electrode groups may also be selected. A combination of the electrode portion pair used for the pulse wave measurement increases, whereby higher precision volume pulse wave measurement can be performed and the degree of freedom in the attachment position of the pulse wave measurement electrode unit during measurement increases.

In the case shown in FIG. 20, the first electrode portion 20_{EG1} of the first electrode group EG1 and the first electrode portion 20_{EG2} of the second electrode group EG2 are simultaneously selected as the specific first electrode portion by switching the switches SW11, SW12, the first current application electrode 20A_{EG1} included in the first electrode portion 20_{EG1} of the first electrode group EG1 and the first current application electrode 20A_{EG2} included in the first electrode portion 20_{EG2} of the second electrode group EG2 are simultaneously connected to the constant current supply section 110, and the first voltage measurement electrode 20B_{EG1} included in the first electrode portion 20_{EG1} of the first electrode group EG1 and the first voltage measurement electrode 20B_{EG2} included in the first electrode portion 20_{EG2} of the second electrode group EG2 are simultaneously connected to the impedance measurement section 120. The second electrode portion 30_{EG1} of the first electrode group EG1 and the second electrode portion 30_{EG2} of the second electrode group EG2 are simultaneously selected as the specific second electrode portion by switching the switches SW21, SW22, the second current application electrode 20B_{EG1} included in the second electrode portion 30_{EG1} of the first electrode group EG1 and the second current application electrode 20B_{EG2} included in the second electrode portion 30_{EG2} of the second electrode group EG2 are simultaneously connected to the constant current supply section 110, and the second voltage measurement electrode 30B_{EG1} included in the second electrode portion 30_{EG1} of the first electrode group EG1 and the second voltage measurement electrode 30B_{EG2} included in the second electrode portion 30_{EG2} of the second electrode group EG2 are simultaneously connected to the impedance measurement section 120. Thus, the volume pulse wave measurement can be performed by simultaneously selecting two first electrode portions and second electrode portions adjacent to the skin immediately above the radial artery 510 as the electrodes for pulse wave measurement, and performing the measurement of volume pulse wave.

The selection of the first electrode portion by the switches SW11, SW12 is not necessarily limited to selecting a single first electrode portion, and a plurality of adjacent first electrode portions may be simultaneously selected. In addition, the selection of the second electrode portion by the switches SW21, SW22 is not necessarily limited to selecting a single second electrode portion, and a plurality of adjacent second electrode portions may be simultaneously selected. Thus, the combination of the electrode portion pair used in the pulse wave measurement increases, and the degree of freedom in the attachment position of the pulse wave measurement electrode unit during measurement increases.

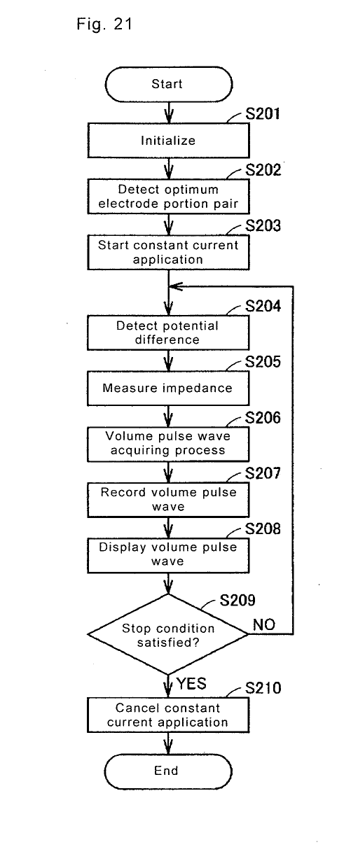

A processing procedure of the pulse wave measurement device 100E of when actually making the determination of an optimum electrode portion pair by performing various switching of the electrode portion will now be described. FIG. 21 is a flowchart showing a flow of the processing procedure of the pulse wave measurement device. A program complying with the flowchart is stored in the memory section 140 shown in FIG. 17 in advance, and the process proceeds by having the CPU 130 read out and execute the program from the memory section 140.

As shown in FIG. 21, when the subject operates the operation section 160 of the pulse wave measurement device 100E to input the command of power ON, power serving as a power supply is supplied from the power supply section 170 to the CPU 130 thereby driving the CPU 130, and initialization of the pulse wave measurement device 100E is performed (step S201). The subject positions and attaches in advance the pulse wave measurement electrode unit 10C described above at a predetermined position of the wrist.

The subject then operates the operation button of the operation section 160 of the pulse wave measurement device 100E to input the command to start measurement, whereby the CPU 130 gives an instruction of the switching selection of the first electrode portion or the second electrode portion to the switches SW11, SW12, SW21, and SW22, performs measurement of the fluctuation of the biological impedance for each combinations of the various electrode portion pairs and determines the combination of the optimum electrode portion pair (step S202). The measurement of the fluctuation of the biological impedance complies with the measurement flow (steps S102 to S106 shown in FIG. 5) described in the first embodiment, and is performed by supplying a constant current between the pair of current application electrodes included in the selected electrode portion pair, and detecting the potential difference between the pair of voltage measurement electrodes included in the selected electrode portion pair in such a case for a predetermined time.

More specifically, in determining the combination of the optimum electrode portion pair, SW11, SW12, SW21, and SW22 are first switched to select the first electrode portion and the second electrode portion included in each of the first to the fourth electrode groups EG1 to EG4 as the electrode portion pair for pulse wave measurement, and the impedance measurement is performed for each combination. The four impedance fluctuation waveforms obtained in such a manner are then compared, where the impedance fluctuation waveform measured with the largest amplitude is stored, and the combination of the first electrode portion and the second electrode portion of the electrode group used for such measurement is stored as an optimum electrode portion pair A.

Then, the switches SW11, SW12, SW21, and SW22 are switched, the different electrode portion of the adjacent electrode groups such as the first electrode portion of the first electrode group EG1 and the second electrode portion of the second electrode group EG2, then the first electrode portion of the second electrode group EG2 and the second electrode portion of the first electrode group EG1, and so on are selected as the electrode portion pair for pulse wave measurement, and the impedance measurement is performed for each combination. A total of six impedance fluctuation waveforms obtained in such a manner are then compared, where the impedance fluctuation waveform measured with the largest amplitude is stored, and the combination of the first electrode portion and the second electrode portion used for such measurement is stored as an optimum electrode portion pair B.

Furthermore, the switches SW11, SW12, SW21, and SW22 are switched, the first electrode portions of the adjacent electrode groups or the second electrode portions of the adjacent electrode groups such as the first electrode portion of the first electrode group EG1 and the first electrode portion of the second electrode group EG2 and the second electrode portion of the first electrode group EG1 and the second electrode portion of the second electrode group EG2, and then the first electrode portion of the second electrode group EG2 and the first electrode portion of the third electrode group EG3 and the second electrode portion of the second electrode group EG2 and the second electrode portion of the third electrode group EG3, and so on are respectively assumed as one electrode portion and selected as the electrode portion pair for pulse wave measurement, and the impedance measurement is performed for each combination. A total of three impedance fluctuation waveforms obtained in such a manner are then compared, where the impedance fluctuation waveform measured with the largest amplitude is stored, and the combination of the first electrode portion and the second electrode portion used for such measurement is stored as an optimum electrode portion pair C.

Thereafter, the three impedance fluctuation waveforms obtained when the three optimum electrode portion pairs A to C are selected are compared, the impedance fluctuation waveform measured with the largest amplitude among them is extracted, and the first electrode portion and the second electrode portion used for such measurement is determined for the combination of the optimum electrode portion pair. The combination of the optimum electrode portion pair is determined in step S202 in the above manner.

The switches SW11, SW12, SW21, and SW22 are then switched such that the combination of the optimum electrode portion pair determined in the above manner is re-selected, and the current application electrode and the voltage measurement electrode included in such optimum electrode portions are respectively connected to the constant current supply section 110 and the impedance measurement section 120. The CPU 130 gives an instruction to start the constant current application to the constant current supply section 110, whereby the constant current supply section 110 supplies the constant current between the selected pair of current application electrodes (step S203). Thereafter, the CPU 130 gives an instruction to detect the potential difference to the impedance measurement section 120, whereby the potential difference between the selected pair of voltage measurement electrodes is detected by the impedance measurement section 120 (step S204), and the biological impedance is measured (step S205). The detected biological impedance is then digitized by the impedance measurement section 120 and inputted to the CPU 130, and the volume pulse wave is acquired by the volume pulse wave acquiring unit 131 (step S206). The acquired volume pulse wave is stored in the memory section 140 as a measurement result (step S207), and then displayed on the display section 150 (step S208). The display section 150 displays the volume pulse wave, for example, as numerical values or waveforms.

The series of operations from step S204 to step S208 are repeatedly performed (when NO in step S209) until a predetermined stop condition (e.g., operation of measurement stop switch by user, elapse of set time by timer circuit, and the like) is satisfied. When the predetermined stop condition is satisfied (when YES in step S209), the CPU 130 gives an instruction to cancel the constant current application to the constant current supply section 110 (step S210). The pulse wave measurement device 100E then takes a standby state, and the supply of power serving as a power supply is stopped after input of command of power OFF by the operation section 160 of the subject is made. Therefore, the volume pulse wave that changes with time can be measured in real time.

In the pulse wave measurement device 100E according to the present embodiment, the volume pulse wave measurement having a high degree of freedom in positioning and high precision can be performed by performing such switching of the electrode portion.

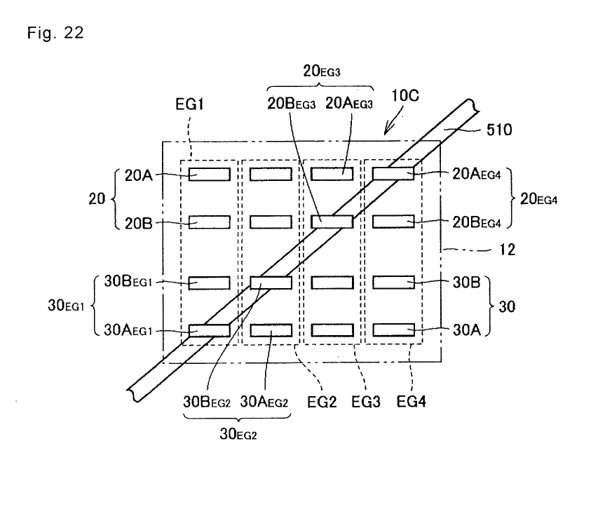

In the above description, a case where the switches SW11, SW12 serving as the first electrode portion selecting unit for switchably selecting a specific first electrode portion and the switches SW21, SW22 serving as the second electrode portion selecting unit for switchably selecting a specific second electrode portion are appropriately switched to select two electrodes included in the same first electrode portion as the first current application electrode and the first voltage measurement electrode, and select two electrodes included in the same second electrode portion as the second current application electrode and the second voltage measurement electrode, thereby enhancing the degree of freedom in position of the pulse wave measurement electrode unit 10C and the wrist has been described. However, the positional relationship of the electrode and the radial artery as shown in FIG. 22 is also assumed. In such a case, the switch SW11 further functions as a first electrode portion current application electrode selecting unit, the switch SW12 further functions as a first electrode portion voltage measurement electrode portion selecting unit, the switch SW21 functions as a second electrode portion current application electrode selecting unit, and the switch SW22 functions as a second electrode portion voltage measurement electrode portion selecting unit, where high precision volume pulse wave measurement is realized by individually and independently switching the four switches SW11, SW12, SW21, and SW22.

In other words, as shown in FIG. 22, the first current application electrode 20A_{EG4} of the first electrode portion 20_{EG4} of the fourth electrode group EG4 is selected as the current application electrode included in a specific first electrode portion by switching the switch SW11, the second current application electrode 30A_{EG1} of the second electrode portion 30_{EG1} of the first electrode group EG1 is selected as the current application electrode included in a specific second electrode portion by switching the switch SW21, and the first current application electrode 20A_{EG4} of the first electrode portion 20_{EG4} of the fourth electrode group EG4 and the second current application electrode 30A_{EG1} of the second electrode portion 30_{EG1} of the first electrode group EG1 are connected to the constant current supply section 110. The first voltage measurement electrode 20B_{EG3} of the first electrode portion 20_{EG3} of the third electrode group EG3 is selected as the voltage measurement electrode included in a specific first electrode portion by switching the switch SW12, the second voltage measurement electrode 30B_{EG2} of the second electrode portion 30_{EG2} of the second electrode group EG2 is selected as the voltage measurement electrode included in a specific second electrode portion by switching the switch SW22, and the first voltage measurement electrode 20B_{EG3} of the first electrode portion 20_{EG3} of the third electrode group EG3 and the second voltage measurement electrode 30B_{EG2} of the second electrode portion 30_{EG2} of the second electrode group EG2 are connected to the impedance measurement section 120. The high precision volume pulse wave measurement can be carried out by selecting the first current application electrode, the first voltage measurement electrode, the second current application electrode, and the second voltage measurement electrode closest to the skin located immediately above the radial artery 510 as the electrodes for pulse wave measurement, and performing the measurement of the volume pulse wave.

Thus, the selection of the first current application electrode, the first voltage measurement electrode, the second current application electrode, and the second voltage measurement electrode by the switches SW11, SW12, SW21, SW22 can be freely selected beyond the framework of the electrode group and the electrode portion. Therefore, the combination of electrodes to use for the pulse wave measurement increases, whereby the volume pulse wave measurement of higher precision can be performed, and the degree of freedom in the attachment position of the pulse wave measurement electrode unit during measurement increases.

### Fifth Embodiment

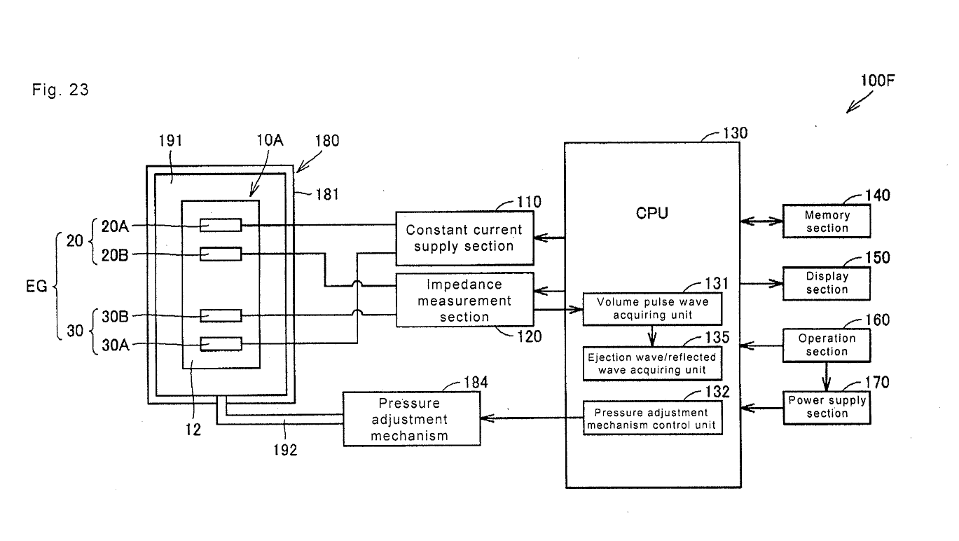

FIG. 23 is a function block diagram showing a configuration of a pulse wave measurement device according to a fifth embodiment of the present invention. First, a configuration of a pulse wave measurement device 100F according to the present embodiment will be described with reference to FIG. 23. In the figure, the same reference symbols are denoted for the portions similar to the pulse wave measurement device 1008 according to the second embodiment, and the description thereof will not be repeated.

As shown in FIG. 23, in the pulse wave measurement device 100F according to the present embodiment, an ejection wave/reflected wave acquiring unit 135 is arranged in the CPU 130. The ejection wave/reflected wave acquiring unit 135 analyzes the volume pulse wave based on the information of the volume pulse wave obtained in the volume pulse wave acquiring unit 131 to calculate at least one of the ejection wave or the reflected wave of the radial artery 510.

The ejection wave is the pulse wave component generated when the heart contracts, and the pulse wave component generated when the ejection wave is reflected at each area of the artery is the reflected wave. The AI (Augmentation Index) derived from the ejection wave and the reflected wave is known to be an index correlated with distensibility of the artery and a degree of a cardiac load.

In order to calculate the ejection wave or the reflected wave at high precision, it is essential that the volume pulse wave obtained by the volume pulse acquiring unit 131 is measured at high precision. Thus, the pulse wave measurement device 100G according to the present embodiment is equipped with a compression mechanism including the air bag 191 and the pressure adjustment mechanism 184, similar to the pulse wave measurement device 100B according to the second embodiment, and is configured to enable measurement of the volume pulse wave at the maximum amplitude by such a compression mechanism.

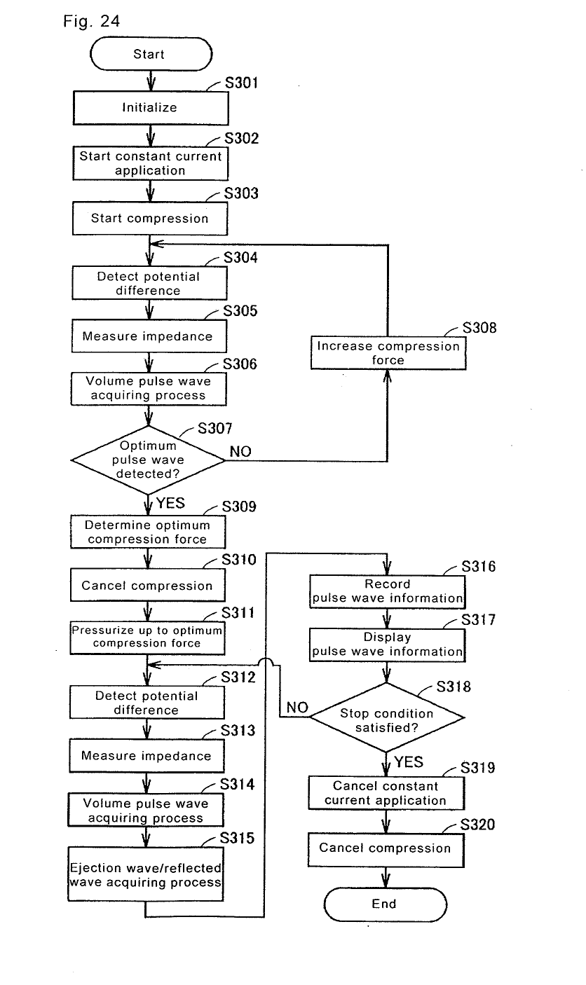

FIG. 24 is a flowchart showing a processing procedure of the pulse wave measurement device according to the present embodiment. The processing procedure in the pulse wave measurement device 100F according to the present embodiment will now be described with reference to FIG. 24. The program complying with the flowchart is stored in advance in the memory section 140 shown in FIG. 23, and the process proceeds by having the CPU 130 read out and execute the program from the memory section 140.

As shown in FIG. 24, when the subject operates the operation section 160 of the pulse wave measurement device 100F to input the command of power ON, power serving as a power supply is supplied from the power supply section 170 to the CPU 130 thereby driving the CPU 130, and initialization of the pulse wave measurement device 100A is performed (step S301). The subject positions and attaches in advance the cuff 180 described above at a predetermined position of the wrist.

The subject then operates the operation button of the operation section 160 of the pulse wave measurement device 100F to input the command to start measurement, whereby the CPU 130 gives an instruction to start constant current application to the constant current supply section 110. The constant current supply section 110 then supplies constant current between the pair of current application electrodes 20A, 30A (step S302). Thereafter, the pressure adjustment mechanism 184 is driven by the pressure adjustment mechanism control unit 132 arranged in the CPU 130, the air is fed to the air bag 191 arranged in the cuff 180, and the compression of the radial artery is started at a predetermined level (step S303). Next, the CPU 130 gives an instruction to detect the potential difference to the impedance measurement section 120. The potential difference between the pair of voltage measurement electrodes 20B, 30B is detected by the impedance measurement section 120 for a predetermined time (step S304), and the fluctuation of the biological impedance is measured (step S305). The fluctuation information of the detected biological impedance is digitized by the impedance measurement section 120 and inputted to the CPU 130, and the volume pulse wave is acquired in the volume pulse wave acquiring unit 131 (step S306).

The CPU 130 then determines in step S307 whether the amplitude of the measured volume pulse wave is a magnitude suited for the calculation of the ejection wave/reflected wave, proceeds to step S308 if determined that the magnitude of the amplitude is insufficient (when NO in step S307) and increases the compression force with respect of the radial artery by a predetermined level, and returns to step S304. If determined that the magnitude of the amplitude is sufficient (when YES in step S307), the CPU 130 proceeds to step S309, and determines the cuff pressure as the cuff pressure at which the optimum compression force is obtained.

Thereafter, the CPU 130 outputs a command of rapid exhaust to the pressure adjustment mechanism 184 to once cancel the compression of the radial artery by the compression mechanism (step S310), and again drives the pressure adjustment mechanism 184 to expand the air bag 191 to the cuff pressure at which the optimum compression force determined in step S309 is obtained (step S311). The CPU 130 then outputs a command to detect the potential difference to the impedance measurement section 120, whereby the potential difference between the pair of voltage measurement electrodes 20B, 30B is detected in the impedance measurement section 120 (step S312), and the biological impedance is measured (step S313). The detected biological impedance is then digitized by the impedance measurement section 120 and inputted to the CPU 130, and the volume pulse wave is acquired in the volume pulse wave acquiring unit 131 (step S314). The acquired volume pulse wave is inputted to the ejection wave/reflected wave acquiring unit 135, and the ejection wave or/and the reflected wave is calculated in the ejection wave/reflected wave acquiring unit 135 (step S315). The pulse wave information including the acquired volume pulse wave and the calculated ejection wave or/and the reflected wave is stored in the memory section 140 as a measurement result (step S316), and thereafter displayed on the display section 150 (step S317). The display section 150 displays the volume pulse wave or the ejection wave or/and the reflected wave, for example, as numerical values or waveforms.

The series of operations from step S312 to step S317 are repeatedly performed (when NO in step S318) until a predetermined stop condition (e.g., operation of measurement stop switch by user, elapse of set time by timer circuit, and the like) is satisfied. When the predetermined stop condition is satisfied (when YES in step S318), the CPU 130 gives an instruction to cancel the constant current application to the constant current supply section 110 (step S319). The CPU 130 then outputs a command of rapid exhaust to the pressure adjustment mechanism 184 to cancel the compression of the radial artery by the compression mechanism (step S319). The pulse wave measurement device 100F then takes a standby state, and the supply of power serving as a power supply is stopped after input of command of power OFF by the operation section 160 of the subject is made. Therefore, the volume pulse wave and the ejection wave or/and the reflected wave that changes with time can be measured in real time.

According to the pulse wave measurement device 100F described above, the pulse wave measurement device capable of measuring the ejection wave and the reflected wave at high precision can be obtained. The pulse wave measurement device for measuring the pressure pulse wave using the tonometry method has been normally used for the conventional pulse wave measurement device capable of measuring the ejection wave and the reflected wave. In the pulse wave measurement device adopting the tonometry method, the measuring site needed to be pressed until a flat portion forms at a blood vessel wall of the artery in measuring the pulse wave, and thus a fixing mechanism for immovably fixing the measuring site, a positioning mechanism for reliably compressing the artery, and the like are necessary. Through the adoption of the configuration of the present embodiment, on the other hand, the pulse wave measurement device capable of easily measuring the ejection wave and the reflected wave without including a complex mechanism can be structured, and a high performance pulse wave measurement device can be inexpensively provided.

### Sixth Embodiment

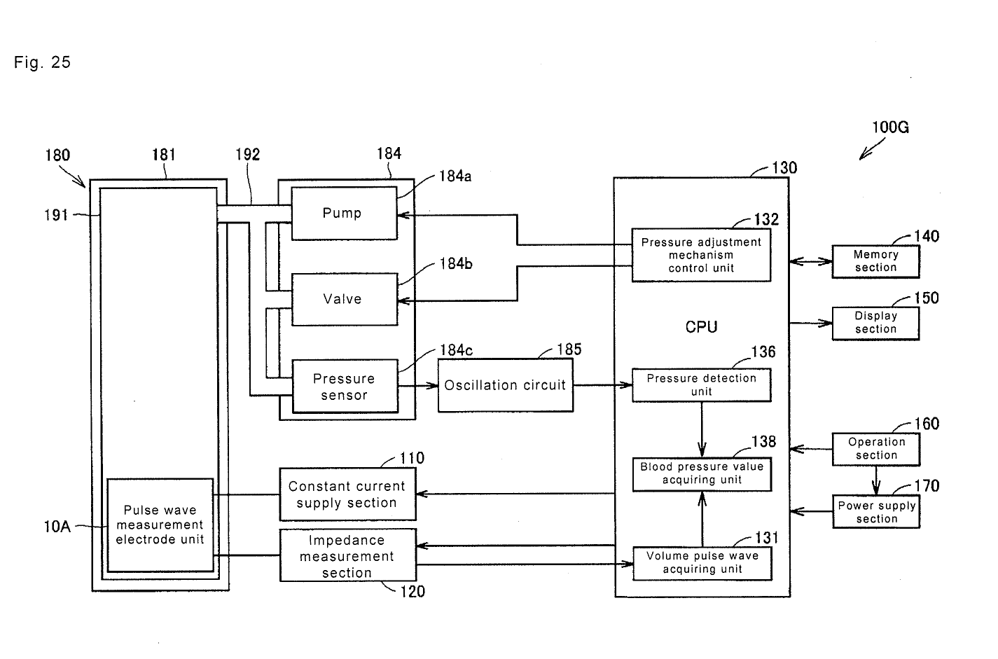

FIG. 25 is a function block diagram showing a configuration of a pulse wave measurement device according to a sixth embodiment of the present invention. A configuration of a pulse wave measurement device 100G according to the present embodiment will be described below with reference to FIG. 25. In the figure, the same reference symbols are denoted for the portions similar to the pulse wave measurement device 1008 according to the second embodiment, and the description thereof will not be repeated.

The pulse wave measurement device 100G according to the present embodiment is a pulse wave measurement device having a blood pressure value acquiring function of a capacity vibration method. As shown in FIG. 25, in the pulse wave measurement device 100G according to the present embodiment, a pressure detection unit 136 and a blood pressure value acquiring unit 138 are arranged in the CPU 130. The pressure detection unit 136 corresponds to a compression force detection unit for detecting the compression force on the artery by detecting the cuff pressure based on the information outputted from the pressure sensor 184c described below. The blood pressure value acquiring unit 138 acquires a systolic blood pressure value (maximum blood pressure value) and a diastolic blood pressure value (minimum blood pressure value) based on the information of the volume pulse wave obtained in the volume pulse wave acquiring unit 131 and the cuff pressure information obtained in the pressure detection unit 136.

The systolic blood pressure value and the diastolic blood pressure value are blood pressure values measured at a point the pulsation of the artery significantly changes in the process of fluctuating the compression force by the cuff, and are conventionally known as typical indices of health management.

The pulse wave measurement device 100G according to the present embodiment includes a compression mechanism substantially similar to the compression mechanism described in the pulse wave measurement device 1008 in the second embodiment, and realizes the compression force fluctuation (i.e., cuff pressure fluctuation) of the cuff using the compression mechanism, and acquires the volume pulse wave while detecting the cuff pressure to acquire the systolic blood pressure value and the diastolic blood pressure value in the blood pressure value acquiring unit 138 based thereon.

More specifically, as shown in FIG. 25, the pulse wave measurement device 100G according to the present embodiment is equipped with a compression mechanism including a cuff 180 with the air bag 191 and the cuff cover 181 including the air bag 191, and the pressure adjustment mechanism 184 for adjusting the internal pressure (cuff pressure) of the air bag 191, where the pressure adjustment mechanism 184 includes a pump 184a, a valve 184b, and a pressure sensor 184c. The CPU 130 is equipped with the pressure adjustment mechanism control unit 132 for controlling the pressure adjustment mechanism 184, where the pressure adjustment mechanism control unit 132 is configured by a pump drive circuit for driving the pump, a valve drive circuit for driving the valve, and the like. The cuff pressure information detected by the pressure sensor 184c is inputted to the pressure detection unit 136 of the CPU 130 through an oscillation circuit 185, and the like.

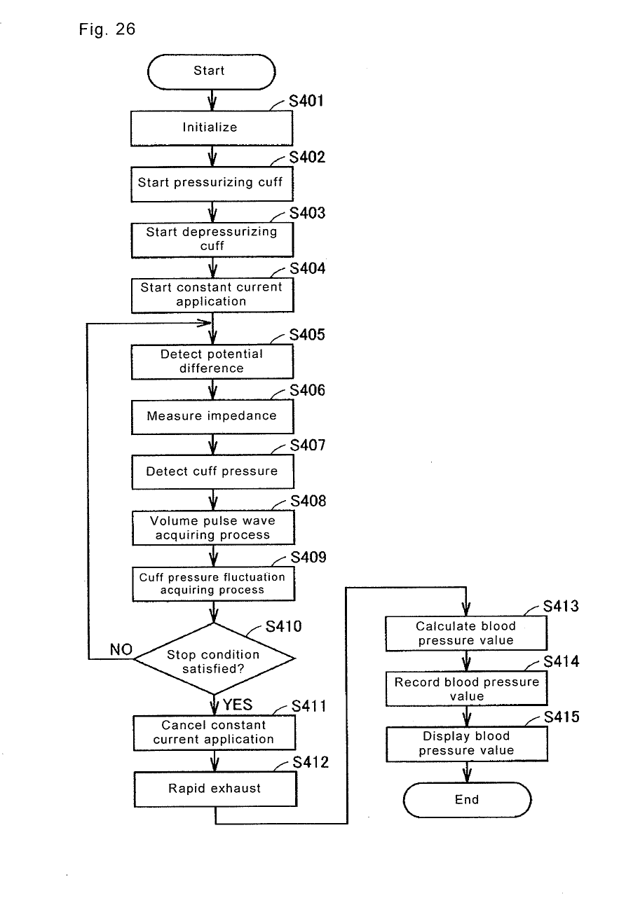

FIG. 26 is a flowchart showing a processing procedure of the pulse wave measurement device according to the present embodiment. The processing procedure in the pulse wave measurement device 100G according to the present embodiment will now be described with reference to FIG. 26. The program complying with the flowchart is stored in advance in the memory section 140 shown in FIG. 25, and the process proceeds by having the CPU 130 read out and execute the program from the memory section 140.

As shown in FIG. 26, when the subject operates the operation section 160 of the pulse wave measurement device 100G to input the command of power ON, power serving as a power supply is supplied from the power supply section 170 to the CPU 130 thereby driving the CPU 130, and initialization of the pulse wave measurement device 100A is performed (step S401). The subject positions and attaches in advance the cuff 180 described above at a predetermined position of the wrist.

The subject then operates the operation button of the operation section 160 of the pulse wave measurement device 100G to input the command to start measurement, so that the pump 184a is driven by the pressure adjustment mechanism control unit 132 arranged in the CPU 130, and the air is fed to the air bag 191 arranged in the cuff 180 to gradually raise the cuff pressure (step S402). The cuff pressure is detected by the pressure sensor 184c, where when detected that the cuff pressure reached to a predetermined level, the CPU 130 stops the pump 184a, gradually opens the closed valve 184b, and gradually exhausts the air of the air bag 191 to gradually depressurize the cuff pressure (step S403).

In the process of depressurizing the cuff pressure at a fine speed, the CPU 130 gives an instruction to start constant current application to the constant current supply section 110, whereby the constant current supply section 110 then supplies the constant current between the pair of current application electrodes 20A, 30A (step S404). The CPU 130 then gives an instruction to detect the potential difference to the impedance measurement section 120, whereby the potential difference between the pair of voltage measurement electrodes 20B, 30B is detected by the impedance measurement section 120 (step S405), and the biological impedance is calculated (step S406). The CPU 130 then detects the pressure information outputted from the pressure sensor 184c via the oscillation circuit 185 (step S407). The detected biological impedance is digitized by the impedance measurement section 120 and inputted to the CPU 130, and the pressure information is inputted to the CPU 130 from the pressure sensor 184c via the oscillation circuit 185, so that the volume pulse wave is acquired in the volume pulse wave acquiring unit 131 and the fluctuation information of the cuff pressure is respectively acquired in the pressure detection unit 136 (steps S408, S409).

The series of operations from step S405 to step S409 are repeatedly performed (when NO in step S410) until a predetermined stop condition (e.g., elapse of set time by timer circuit, cuff pressure is depressurized to a predetermined level, or the like) is satisfied. When the predetermined stop condition is satisfied (when YES in step S410), the CPU 130 gives an instruction to cancel the constant current application to the constant current supply section 110 (step S411).

The CPU 130 then outputs a command of rapid exhaust to the pressure adjustment mechanism 184 to cancel the compression of the radial artery by the compression mechanism (step S412), inputs the volume pulse wave obtained in step S408 to the blood pressure value acquiring unit 138 and inputs the cuff pressure fluctuation information obtained in step S409 to the blood pressure value acquiring unit 138 to acquire the systolic blood pressure value and the diastolic blood pressure value (step S413). The blood pressure value acquiring unit 138 extracts a point where the amplitude of the volume pulse wave significantly changes in the process of fluctuating the compression force by the cuff, and references the cuff pressure at the time to acquire the systolic blood pressure value and the diastolic blood pressure value. Subsequently, the systolic blood pressure value and the diastolic blood pressure value acquired in the blood pressure value acquiring unit 138 are stored in the memory section 140 as a measurement result (step S414), and then the measurement result is displayed by the display section 150 (step S415). The display section 150 displays the systolic blood pressure value and the diastolic blood pressure value, for example, as numerical values. After recording and displaying the blood pressure value information, the pulse wave measurement device 100G takes a standby state, and the supply of power serving as a power supply is stopped after input of command of power OFF by the operation section 160 of the subject is made.

According to the pulse wave measurement device 100G described above, the pulse wave measurement device capable of measuring the systolic blood pressure value and the diastolic blood pressure value at high precision can be obtained. In a conventional sphygmomanometer of an oscillometric type, a pressure pulse wave is obtained from the fluctuation of the cuff pressure, and the systolic blood pressure value and the diastolic blood pressure are acquired from the pressure pulse wave. However, when such a method is adopted, a large difference in compression force with respect to the measuring site is created between the end and the central part of the cuff in compressing the measuring site by the cuff, as described above, thus it is difficult to evenly compress the measuring site, and the pulse wave measurement of high precision becomes difficult. Furthermore, when the site where plural arteries are running is adopted for the measuring site, the pulse waves of the plural arteries are averaged and detected and thus the high precision pulse wave measurement becomes difficult. By adopting the configuration of the present embodiment, all of the above problems are solved, and a pulse wave measurement device capable of acquiring the blood pressure value at satisfactory precision can be obtained.

### Seventh Embodiment

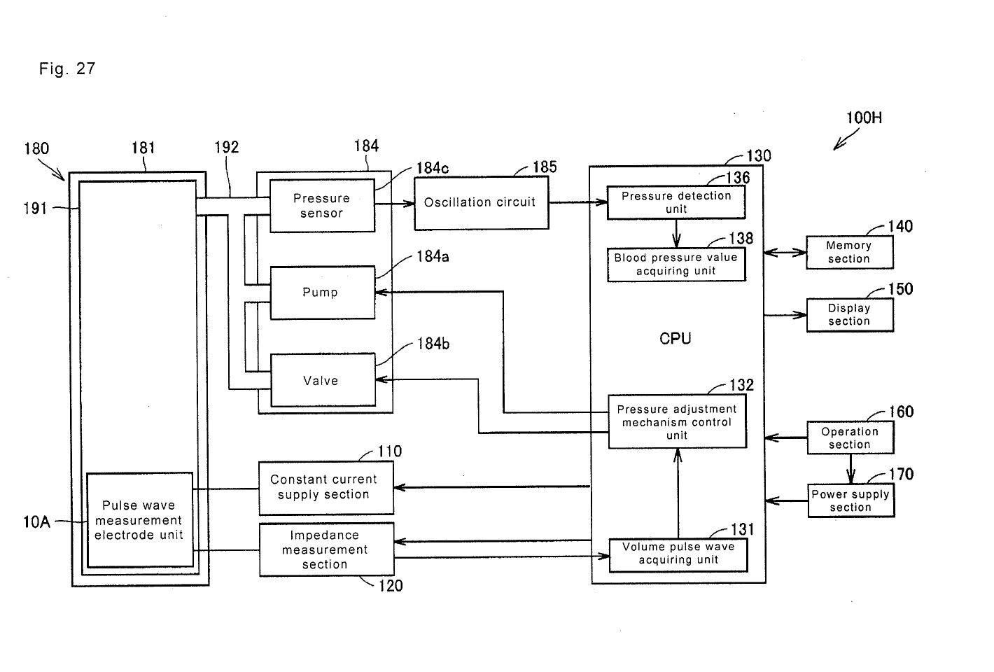

FIG. 27 is a function block diagram showing a configuration of a pulse wave measurement device according to a seventh embodiment of the present invention. A configuration of a pulse wave measurement device 100H according to the present embodiment will be described below with reference to FIG. 27. In the figure, the same reference symbols are denoted for the portions similar to the pulse wave measurement device 1008 according to the second embodiment, and the description thereof will not be repeated.

The pulse wave measurement device 100H according to the present embodiment is a pulse wave measurement device having a blood pressure value acquiring function using a capacity compensation method. As shown in FIG. 27, in the pulse wave measurement device 100H according to the present embodiment, the pressure detection unit 136 and the blood pressure value acquiring unit 138 are arranged in the CPU 130. The pressure detection unit 136 corresponds to a compression force detection unit for detecting the compression force on the artery by detecting the cuff pressure based on the information outputted from the pressure sensor 184c described below. The blood pressure value acquiring unit 138 acquires the systolic blood pressure value (maximum blood pressure value) and the diastolic blood pressure value (minimum blood pressure value) based on the cuff pressure information obtained in the pressure detection unit 136.

In the volume compensation method, the cuff pressure is servo controlled so that an equilibrium between an inner pressure (pressure generated by pumping function of the heart, that is, blood pressure) applied on the blood vessel wall of the artery and an outer pressure (compression force by cuff) is always achieved, and the systolic blood pressure value and the diastolic blood pressure value are acquired by detecting the cuff pressure at the time.

The pulse wave measurement device 100G according to the present embodiment includes a compression mechanism substantially similar to the compression mechanism described in the pulse wave measurement device 1006 in the second embodiment, and a servo control of the cuff pressure is performed using the compression mechanism. The pulse wave measurement electrode unit according to the present invention is used for the setting of the target value of servo control in this case, and the determination on whether the inner pressure applied on the blood vessel wall and the outer pressure are in an equilibrium state by the servo control.

More specifically, as shown in FIG. 27, the pulse wave measurement device 100H according to the present embodiment is equipped with a compression mechanism including a cuff 180 with the air bag 191 and the cuff cover 181 including the air bag 191, and the pressure adjustment mechanism 184 for adjusting the internal pressure (cuff pressure) of the air bag 191, where the pressure adjustment mechanism 184 includes the pump 184a, the valve 184b, and the pressure sensor 184c. The CPU 130 is equipped with the pressure adjustment mechanism control unit 132 for controlling the pressure adjustment mechanism 184, where the pressure adjustment mechanism control unit 132 is configured by a pump drive circuit for driving the pump, a valve drive circuit for driving the valve, and the like. The cuff pressure information detected by the pressure sensor 184c is inputted to the pressure detection unit 136 of the CPU 130 through the oscillation circuit 185, and the like.

In the pulse wave measurement device 100H according to the present embodiment, the pressure adjustment mechanism control unit 132 performs the servo control of the cuff pressure based on the volume pulse wave information acquired in the volume pulse wave acquiring unit 131, as opposed to the pulse wave measurement device 100G having the blood pressure value acquiring function of an oscillometric type in the sixth embodiment. The systolic blood pressure value and the diastolic blood pressure value are acquired based on the cuff pressure information obtained by the pressure sensor 184c.

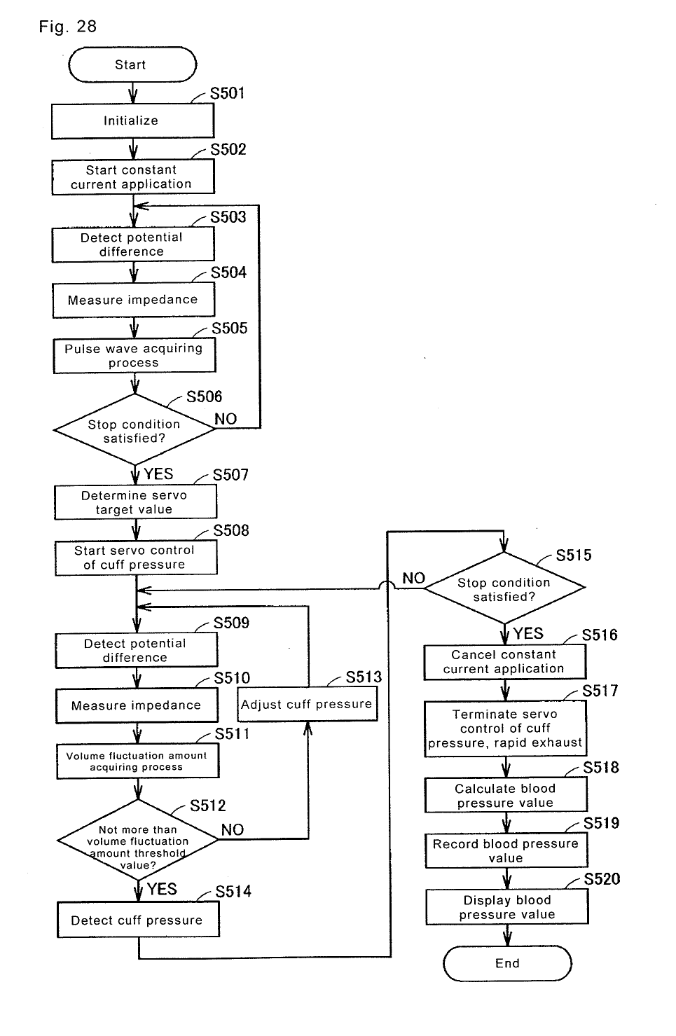

FIG. 28 is a flowchart showing a processing procedure of the pulse wave measurement device according to the present embodiment. The processing procedure in the pulse wave measurement device 100H according to the present embodiment will now be described with reference to FIG. 28. The program complying with the flowchart is stored in advance in the memory section 140 shown in FIG. 27, and the process proceeds by having the CPU 130 read out and execute the program from the memory section 140.

As shown in FIG. 28, when the subject operates the operation section 160 of the pulse wave measurement device 100H to input the command of power ON, power serving as a power supply is supplied from the power supply section 170 to the CPU 130 thereby driving the CPU 130, and initialization of the pulse wave measurement device 100A is performed (step S501). The subject positions and attaches in advance the cuff 180 described above at a predetermined position of the wrist.

When the subject operates the operation button of the operation section 160 of the pulse wave measurement device 100H to input the command to start measurement, the CPU 130 gives an instruction to start the constant current application to the constant current supply section 110, whereby the constant current supply section 110 supplies constant current between the pair of current application electrodes 20A, 30A (step S502). Subsequently, the CPU 130 gives an instruction to detect the potential difference to the impedance measurement section 120, whereby the potential difference between the pair of voltage measurement electrodes 20B, 30B is detected by the impedance measurement section 120 (step S503), and the biological impedance is calculated (step S504). The detected biological impedance is then digitized by the impedance measurement section 120 and inputted to the CPU 130, and the volume pulse wave is acquired in the volume pulse wave acquiring unit 131 (step S505).

The series of operations from step S503 to step S505 are repeatedly performed (when NO in step S506) until a predetermined stop condition (e.g., operation of measurement stop switch by user, elapse of set time by timer circuit, and the like) is satisfied. When the predetermined stop condition is satisfied (when YES in step S506), the CPU 130 determines an initial control target value of the cuff pressure based on the information of the measured volume pulse wave (step S507).

The pump 184a is then driven by the pressure adjustment mechanism control unit 132 arranged in the CPU 130, and the air is fed to the air bag 191 arranged in the cuff 180 to start the servo control of the cuff pressure (step S508). When the cuff pressure reaches the initial control target value, the CPU 130 gives an instruction to detect the potential difference to the impedance measurement section 120. The potential difference between the pair of voltage measurement electrodes 20B, 30B is detected by the impedance measurement section 120 (step S509), and the fluctuation of the biological impedance is measured (step S510). The detected biological impedance is then digitized by the impedance measurement section 120 and inputted to the CPU 130, and the volume fluctuation amount is acquired (step S511). Thereafter, in step S512, whether the acquired volume fluctuation amount is not more than a predefined threshold value is determined, where if it is not determined that the volume fluctuation amount is not more than the threshold value (when NO in step S512), the cuff pressure adjustment (change of servo target value, servo control of the cuff pressure towards the servo target value after change, and the like) is performed based on the artery volume signal derived therefrom (step S513), and the process returns from step S509 to step S512, and the detection of the potential difference, the impedance measurement, the acquisition of the volume fluctuation amount, and the determination on whether the volume fluctuation amount is not more than the threshold value are repeated. If determined that the volume fluctuation amount is not more than the predefined threshold value (when YES in step S512), the process proceeds to step S514, the cuff pressure is detected by the pressure sensor 184c, and such information is inputted to the pressure detection unit 136 of the CPU 130 via the oscillation circuit 185.

The series of operations from step S509 to step S514 are repeatedly performed (when NO in step S515) until a predetermined stop condition (e.g., operation of measurement stop switch by user, elapse of set time by timer circuit, and the like) is satisfied. When the predetermined stop condition is satisfied (when YES in step S515), the CPU 130 gives an instruction to cancel the constant current application to the constant current supply section 110 (step S516).

Thereafter, the CPU 130 outputs a command of rapid exhaust to the pressure adjustment mechanism 184, stops the servo control of the cuff pressure and cancels the compression of the radial artery (step S517), and inputs the cuff pressure information obtained in step S514 to the blood pressure value acquiring unit 138 to acquire the systolic blood pressure value and the diastolic blood pressure value (step S518). The systolic blood pressure value and the diastolic blood pressure value acquired in the blood pressure value acquiring unit 138 are then stored in the memory section 140 as a measurement result (step S519), and then the measurement result is displayed by the display section 150 (step S520). The display section 150 displays the systolic blood pressure value and the diastolic blood pressure value as a graph of numerical values or temporal change in values. After recording and displaying the blood pressure value information, the pulse wave measurement device 100H takes a standby state, and the supply of power serving as a power supply is stopped after input of command of power OFF by the operation section 160 of the subject is made.

According to the pulse wave measurement device 100H described above, the pulse wave measurement device capable of measuring the systolic blood pressure value and the diastolic blood pressure value at high precision can be obtained. An optical sensor is used for acquiring the volume pulse wave in the pulse wave measurement device having the blood pressure value acquiring function using the conventional volume compensation method. However, in the pulse wave measurement device using the optical sensor, light emitted from a light emitting element needs to be accurately captured by a light receiving element, which arises problems such as a need to enhance the positioning accuracy. According to the pulse wave measurement device of the present embodiment, the degree of freedom in positioning the electrodes is high, the manufacturing is facilitated, the degree of freedom in positioning and attaching the pulse wave measurement electrode unit to the wrist is high, and excellent convenience is obtained.

In the first to the seventh embodiments described above, the description is made using a case of adopting the wrist for the measuring site, but it should be recognized that the present invention can be applied to the pulse wave measurement device that uses other sites of the body for the measuring site. Other sites of the body that may be adopted for the measuring site includes other sites of four limbs such as an upper arm, an ankle, and a thigh, a neck, a finger, and the like. When a site other than the wrist is adopted for the measuring site, it is desirable that the electrode width W, the inter-electrode portion distance D, and the like are appropriately changed according to the shape and the like of the measuring site.

In the fourth embodiment described above, description is made using the pulse wave measurement electrode unit including four sets of electrode groups, but the number is not particularly limited, and can be appropriately changed in a range of about two to ten sets.

Furthermore, the characteristic configurations disclosed in the first to the seventh embodiments described above can be combined with each other, for example, the pulse wave measurement electrode unit disclosed in the fourth embodiment may be applied to the pulse wave measurement device disclosed in the fifth to the seventh embodiments.

The embodiments disclosed herein are illustrative in all aspects and should not be construed as being restrictive. The technical scope of the present invention is defined by the claims, and all modifications are intended to be encompassed within the meaning and the scope equivalent to the claims.

1. A pulse wave measurement electrode unit for acquiring a volume pulse wave of an artery of a plurality of arteries of a living body by measuring a fluctuation of a biological impedance, the pulse wave measurement electrode unit comprising:
an electrode group including a pair of current application electrodes and a pair of voltage measurement electrodes, the electrode group to be brought into contact with a body surface of the living body during measurement; and
a supporting member configured to support the electrode group, wherein
the electrode group includes a first electrode portion including one of the pair of current application electrodes and one of the pair of voltage measurement electrodes and (ii) a second electrode portion, positioned spaced apart from the first electrode portion, including the other one of the pair of current application electrodes and the other one of the pair of voltage measurement electrodes,
the supporting member supports the electrode group such that (I) a contacting surface with respect to the living body of the first electrode portion and a contacting surface with respect to the living body of the second electrode portion are arranged substantially on an identical plane and (ii) the first electrode portion and the second electrode portion are arranged lined in a direction the artery extends when the pulse wave measurement electrode unit is brought into contact with the body surface of the living body during measurement, and
each of the pair of current application electrodes and each of the pair of voltage measurement electrodes have widths adapted to be arranged across a portion of the body surface located immediately above the artery such that only the artery of the plurality of arteries of the living body is measured.

2. The pulse wave measurement electrode unit of claim 1, wherein
the first electrode portion includes a single electrode used as the one of the pair of current application electrodes and the one of the pair of voltage measurement electrodes, and
the second electrode portion includes a single electrode used as the other one of the pair of current application electrodes and the other one of the pair of voltage measurement electrodes.

3. The pulse wave measurement electrode unit of claim 1, wherein
the first electrode portion includes two electrodes in which the one of the pair of current application electrodes and the one of the pair of voltage measurement electrodes are separated and independent, and
the second electrode portion includes two electrodes in which the other one of the pair of current application electrodes and the other one of the pair of voltage measurement electrodes are separated and independent.

4. The pulse wave measurement electrode unit of claim 3, wherein
a contacting surface of each of the pair of current application electrodes and the pair of voltage measurement electrodes with the body surface of the living body has a substantially rectangular shape in plan view, and
a length of each of the pair of current application electrodes in a direction intersecting a direction the first electrode portion and the second electrode portion are lined is equal to or less than a length of each of the pair of voltage measurement electrodes in the direction intersecting the direction the first electrode portion and the second electrode portion are lined.

5. A pulse wave measurement device comprising:
the pulse wave measurement electrode unit of claim 1;
a constant current supply section configured to supply a constant current between the pair of current application electrodes;
an impedance measurement section configured to measure a fluctuation of a biological impedance by detecting a potential difference created between the pair of voltage measurement electrodes; and
a volume pulse wave acquiring unit configured to acquire a volume pulse wave of the artery based on information obtained by the impedance measurement section.

6. The pulse wave measurement device of claim 5, further comprising:
a compression mechanism configured to press the body surface to compress the artery,
wherein the pulse wave measurement electrode unit is arranged on a compressing surface of the compression mechanism.

7. The pulse wave measurement device of claim 6, wherein the compression mechanism includes (i) a first compression mechanism configured to press a portion arranged with the first electrode portion and the second electrode portion of the supporting member towards the living body and (ii) a second compression mechanism configured to press a portion positioned between the first electrode portion and the second electrode portion of the supporting member towards the living body.

8. The pulse wave measurement device of claim 5, further comprising an ejection wave/reflected wave acquiring unit configured to acquire at least one of an ejection wave and a reflected wave based on information of the volume pulse wave obtained in the volume pulse wave acquiring unit.

9. The pulse wave measurement device of claim 5, further comprising:
a compression mechanism configured to press the body surface to compress the artery;
a compression force detection unit configured to detect a compression force on the artery by the compression mechanism; and
a blood pressure value acquiring unit configured to acquire a diastolic blood pressure value and a systolic blood pressure value based on information of the volume pulse wave obtained in the volume pulse wave acquiring unit and information of the compression force obtained in the compression force detection unit.

10. The pulse wave measurement device of claim 5, further comprising:
a compression mechanism configured to press the body surface to compress the artery;
a compression force control unit configured to control a compression force on the artery by the compression mechanism based on information of the volume pulse wave obtained in the volume pulse wave acquiring unit;
a compression force detection unit configured to detect a compression force on the artery by the compression mechanism; and
a blood pressure value acquiring unit configured to acquire a diastolic blood pressure value and a systolic blood pressure value based on information of the compression force obtained in the compression force detection unit.

11. The pulse wave measurement electrode unit of claim 1, further comprising:
multiple electrode groups each comprising a first electrode portion and a second electrode portion,
wherein the supporting member supports the electrode groups such that the electrode groups are arranged lined in a direction intersecting a direction the first electrode portions and the second electrode portions are lined.

12. A pulse wave measurement device comprising:
the pulse wave measurement electrode unit of claim 11;
a first electrode portion selecting unit configured to switchably select a first electrode portion of one of the electrode groups included in the pulse wave measurement electrode unit;
a second electrode portion selecting unit configured to switchably select a second electrode portion of one of the electrode groups included in the pulse wave measurement electrode unit;
a constant current supply section configured to supply constant current between current application electrodes included in the specific first electrode portion selected by the first electrode portion selecting unit and the second electrode portion selected by the second electrode portion selecting unit;
an impedance measurement section configured to measure a fluctuation of a biological impedance by detecting a potential difference created between voltage measurement electrodes included in the first electrode portion selected by the first electrode portion selecting unit and the second electrode portion selected by the second electrode portion selecting unit; and
a volume pulse wave acquiring unit configured to acquire a volume pulse wave of the artery based on information obtained in the impedance measurement section.

13. A pulse wave measurement device comprising:
the pulse wave measurement electrode unit of claim 11;
a first electrode portion current application electrode selecting unit configured to switchably select a current application electrode included in a first electrode portion of one of the electrode groups included in the pulse wave measurement electrode unit;
a first electrode portion voltage measurement electrode selecting unit configured to switchably select a voltage measurement electrode included in a first electrode portion of one of the electrode groups included in the pulse wave measurement electrode unit;
a second electrode portion current application electrode selecting unit configured to switchably select a current application electrode included in a second electrode portion of one of the electrode groups included in the pulse wave measurement electrode unit;
a second electrode portion voltage measurement electrode selecting unit configured to switchably select a voltage measurement electrode included in a second electrode portion of one of the electrode groups included in the pulse wave measurement electrode unit;
a constant current supply section configured to supply a constant current between the current application electrode selected by the first electrode portion current application electrode selecting unit and the current application electrode selected by the second electrode portion current application electrode selecting unit;
an impedance measurement section configured to measure a fluctuation of a biological impedance by detecting a potential difference created between the voltage measurement electrode selected by the first electrode portion voltage measurement electrode selecting unit and the voltage measurement electrode selected by the second electrode portion voltage measurement electrode selecting unit; and
a volume pulse wave acquiring unit configured to acquire a volume pulse wave of the artery based on information obtained in the impedance measurement section.

14. The pulse wave measurement electrode unit of claim 1, wherein
the pulse wave measurement electrode unit is adapted to attach to a wrist of the living body, and
the supporting member supports the electrode group such that the first electrode portion and the second electrode portion are arranged lined in a direction a radial artery extends through the wrist so that only the radial artery of the radial artery and an ulnar artery extending through the wrist is measured when the pulse wave measurement electrode unit is attached to the wrist.

15. The pulse wave measurement electrode unit of claim 1, wherein a width of each of the pair of current application electrodes and a width of each of the pair of voltage measurement electrodes are both not less than 5 mm and not more than 15 mm.
